(19) United States
(12) Patent Application Publication
(10) Pub. No.: US 2006/0258647 A1
(43) Pub. Date: Nov. 16, 2006

(54) Treatment of maladaptive substance use with cholecystokinin (CCK) antagonists

(75) Inventors: Jennifer Mitchell, Berkeley, CA (US); Howard Fields, Berkeley, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/406,818

(22) Filed: Apr. 18, 2006

Related U.S. Application Data
(60) Provisional application No. 60/677,916, filed on May 4, 2005.

(51) Int. Cl. A61K 31/5513

(52) U.S. Cl. 514/221

(57) ABSTRACT

The present invention provides a method of mitigating a symptom of maladaptive substance use in a subject by inhibiting CCK_{B} receptors. In addition, the invention provides a variety of prescreening and screening methods aimed at identifying agents that modulate a symptom of maladaptive substance use. Methods of the invention can involve assaying test agent binding to CCK_{B} receptors. Alternatively, test agents can be screened for their ability to alter the level of CCK_{B} receptor polypeptides, polynucleotides, or function. Finally, the invention also provides a diagnostic method that entails measuring one or more of these levels and determining risk for maladaptive substance use based on comparison to the corresponding level for a control population.

### CROSS-REFERENCE TO RELATED APPLICATIONS

This application claims priority to and benefit of U.S. Ser. No. 60/677,916, filed May 4, 2005 and is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under grant no. DA001949. The Government may have certain rights in the invention.

### FIELD OF THE INVENTION

This invention pertains to methods of mitigating a symptom of maladaptive substance use, such as symptoms associated with maladaptive use of opioids, psychostimulants, cannabinoids, empathogens, dissociative drugs, and ethanol, as well as to related compositions and screening and diagnostic methods.

### BACKGROUND OF THE INVENTION

Cholecystokinin (CCK) is a neuropeptide that acts in the CNS to modulate appetite, stress, and anxiety (Nemeroff et al., 1978; Schanzer et al., 1978; Willis et al., 1986; Singh et al., 1991; Bhatnagar et al., 2000). Two distinct CCK receptors have been identified: the CCK_{A} (CCK1) receptor and the CCK_{B} (CCK2) receptor (Crawley & Corwin, 1994). Under some circumstances CCK acts as an anti-opioid peptide. For example, CCK attenuates the analgesic effects of morphine and other mu opioid receptor agonists (Faris et al., 1983), while CCK antagonists potentiate the analgesic effects of mu opioid agonists (Watkins et al., 1984; Katsuura & Itoh, 1985). CCK antagonists also impede the acquisition of morphine tolerance (Tang et al., 1984; Dourish et al., 1988) yet have no effect on morphine dependence and withdrawal (Panerai et al., 1987; Baber et al., 1989).

In contrast to its opioid-opposing action in analgesia, there is evidence that CCK contributes to the rewarding effects of opioids, psychostimulants, and ethanol (Higgins et al., 1992; Crespi, 1998). Additionally, a CCK_{B} antagonist blocks both stress-induced reinstatement of cocaine conditioned place preference (Lu et al., 2002) and drug-primed reinstatement of morphine conditioned place preference (Lu et al., 2001). Importantly, in the absence of drug associated cues and stress, CCK_{B} antagonists typically have no effect on place preference (Higgins et al., 1992; Lavigne et al., 1992). Although the contribution of CCK to the acquisition of morphine CPP has been studied (Higgins et al., 1992) its role in the expression of established morphine CPP has not.

There is evidence suggesting that CCK interactions with dopamine are of critical importance for goal directed behaviors. CCK is colocalized with dopamine in approximately 80% of neurons in the substantia nigra pars compacta (Seroogy et al., 1989) and 40% of neurons in the ventral tegmental area (VTA; Wang et al., 1985). Furthermore, dopaminergic neurons in the VTA that project to the NAcc contain CCK (Hokfelt et al., 1980). There are also CCK-containing projections to the NAcc from the prefrontal cortex (Hokfelt et al., 1988; Brog et al., 1993; You et al., 1998). Finally, systemic morphine administration induces the release of both dopamine and CCK in the NAcc (Hamilton et al., 2000).

The effect of dopamine on NAcc neurons in vitro can be either enhanced or suppressed by application of CCK-8 (White & Wang, 1984; Voigt et al., 1986; Wang & Hu, 1986; Yim & Mogenson, 1991) and microinjection of CCK-8 into the NAcc can both attenuate and potentiate the release of dopamine in vivo (Derrien et al., 1993; Ladurelle et al., 1993). These studies suggest that CCK may act in the NAcc by modulating dopamine.

### SUMMARY OF THE INVENTION

In one embodiment, the invention provides a method of mitigating a symptom of maladaptive substance use, wherein the method entails systemically administering an effective amount of an inhibitor of a cholecystokinin-B (CCK_{B}) receptor to a subject, whereby the symptom of maladaptive substance use is mitigated. In one embodiment, the substance can be a drug selected from the group consisting of an opioid, a psychostimulant, a cannabinoid, an empathogen, a dissociative drug, and ethanol. In another embodiment, the substance can be food in general or a particular type of food. The symptom of maladaptive substance use can include, for example, drug reward, incentive salience for a drug, drug craving, drug seeking, drug consumption, drug reinstatement, stress-induced reinstatement, and drug relapse.

In one embodiment of the method, the CCK_{B} receptor inhibitor includes a CCK_{B} receptor antagonist that inhibits a function of the CCK_{B} receptor. In a variation of this embodiment, the CCK_{B} receptor antagonist is selective for the CCK_{B} receptor. An example of the latter is the CCK_{B} receptor antagonist (3R-(+)-2,3-dihydro-1-methyl-2-oxo-5-phenyl-1H-1,4-benzodiazepin-3-yl)-N′-(3-methylphenyl)urea (L-365,260).

In particular embodiments of the method, the CCK_{B} receptor inhibitor is administered via implantation of a depot formulation including the inhibitor.

In one embodiment, the method of mitigating a symptom of maladaptive substance use includes coadministering an inhibitor of an opioid receptor with the CCK_{B} receptor inhibitor. Generally, the amount of the opioid receptor inhibitor administered is sufficient to reduce the risk of drug overdose in the subject. In a variation of this embodiment, the opioid receptor inhibitor includes an opioid receptor antagonist that inhibits a function of the opioid receptor, such as, e.g., the mu opioid receptor. In a variation of this embodiment, the opioid receptor inhibitor includes an opioid receptor antagonist that is selective for the mu opioid receptor. Exemplary selective mu opioid receptor antagonists include naltrexone, naloxone, CTAP, and CTOP.

The invention also provides a pharmaceutical composition including: (a) an inhibitor of a cholecystokinin-B (CCK_{B}) receptor; and (b) an inhibitor of an opioid receptor. In certain embodiments, (a) the amount of the CCK_{B} receptor inhibitor is sufficient to mitigate a symptom of maladaptive substance use in a subject; and (b) the amount of the opioid receptor inhibitor is sufficient to reduce the risk of drug overdose in the subject.

In one embodiment of the pharmaceutical composition, the CCK_{B} receptor inhibitor includes a CCK_{B} receptor antagonist that inhibits a function of the CCK_{B} receptor. In a variation of this embodiment, the CCK_{B} receptor antagonist is selective for the CCK_{B} receptor. An example of the latter is the CCK_{B} receptor antagonist (3R-(+)-2,3-dihydro-1-methyl-2-oxo-5-phenyl-1H-1,4-benzodiazepin-3-yl)-N′-(3-methylphenyl)urea (L-365,260).

In one embodiment of the pharmaceutical composition, the opioid receptor inhibitor includes an opioid receptor antagonist that inhibits a function of the opioid receptor, such as, e.g., the mu opioid receptor. In a variation of this embodiment, the opioid receptor inhibitor includes an opioid receptor antagonist that is selective for the mu opioid receptor. Exemplary selective mu opioid receptor antagonists include naltrexone, naloxone, CTAP, and CTOP.

In particular embodiments, the pharmaceutical composition includes a depot formulation.

Another aspect of the invention is a method of prescreening for an agent that can modulate a symptom of maladaptive substance use in a subject. The method entails: (a) contacting a test agent with a cholecystokinin-B (CCK_{B}) receptor; and (b) determining whether the test agent specifically binds to the CCK_{B} receptor; and (c) if the test agent specifically binds to the CCK_{B} receptor, selecting the test agent as a potential modulator of a symptom of maladaptive substance use in a subject. In certain embodiments, aimed at screening for selective modulators of the CCK_{B} receptor, the prescreening method additionally includes: (d) contacting a test agent with a cholecystokinin-A (CCK_{A}) receptor; (e) determining whether the test agent specifically binds to the CCK_{A} receptor; and (f) if the test agent preferentially binds to the CCK_{B} receptor over the CCK_{A} receptor, selecting the test agent as a potential modulator of a symptom of maladaptive substance use in a subject.

In another embodiment, prescreening for an agent that can modulate a symptom of maladaptive substance use in a subject is carried out by a method that entails: (a) contacting a test agent with a polynucleotide encoding a cholecystokinin-B (CCK_{B}) receptor; and (b) determining whether the test agent specifically binds to the polynucleotide encoding the CCK_{B} receptor; and (c) if the test agent specifically binds to the polynucleotide encoding the CCK_{B} receptor, selecting the test agent as a potential modulator of a symptom of maladaptive substance use in a subject.

Both of these prescreening methods can additionally include recording any test agent that specifically binds to the CCK_{B} receptor or the polynucleotide, respectively, in a database of candidate agents that may modulate a symptom of maladaptive substance use in a subject. In particular embodiments, the contacting step of these prescreening methods is carried out in vitro.

The invention also provides a method of screening for an agent that can modulate a symptom of maladaptive substance use in a subject. The method entails: (a) contacting a test agent with a cholecystokinin-B (CCK_{B}) receptor; (b) determining whether the test agent acts as an agonist or an antagonist of the CCK_{B} receptor; (c) if the test agent acts as an agonist or antagonist of the CCK_{B} receptor, selecting the test agent as a potential modulator of maladaptive substance use in a subject. In certain embodiments, aimed at screening for selective modulators of the CCK_{B} receptor, the prescreening method additionally includes: (d) contacting a test agent with a cholecystokinin-A (CCK_{A}) receptor; and (e) determining whether the test agent acts as an agonist or antagonist of the CCK_{A} receptor; and (f) if the test agent preferentially acts as an agonist or antagonist of the CCK_{B} receptor over the CCK_{A} receptor, selecting the test agent as a potential modulator of maladaptive substance use in a subject.

The screening method can additionally include recording any test agent that acts as an agonist or antagonist of the CCK_{B} receptor in a database of agents that may modulate maladaptive substance use in a subject. In particular embodiments, the contacting step of this screening method is carried out in vitro.

In one embodiment of the screening method, a test agent is contacted with a cell that expresses a CCK_{B} receptor in the absence of test agent, or with a fraction of the cell; the level of CCK_{B} receptors or of polynucleotides encoding CCK_{B} receptors is determined; and the test agent is selected as a potential modulator of a symptom of maladaptive substance use if the level of CCK_{B} receptors, or of polynucleotides encoding CCK_{B} receptors, is altered. In another embodiment, a test agent is contacted with a cell that expresses a CCK_{B} receptor in the absence of test agent, or with a fraction of the cell; the level of a CCK_{B} receptor function is determined; and the test agent is selected as a potential modulator of a symptom of maladaptive substance use if the level of the CCK_{B} receptor function is altered.

In certain embodiments, the screening method is carried out to determine whether the test agent acts as an antagonist of the CCK_{B} receptor. Test agents that act as an antagonist of the CCK_{B} receptor can be selected as potential inhibitors of a symptom of maladaptive substance use.

The screening method can additionally include combining the selected test agent with a pharmaceutically acceptable carrier. In one embodiment, the ability of the selected test agent to modulate drug reward in an animal model can be measured. In a variation of this embodiment, the animal model tests expression of conditioned place preference.

The invention also provides an in vivo method of screening for an agent that that can modulate maladaptive substance use in a subject, wherein the method entails: (a) selecting a modulator of a cholecystokinin-B (CCK_{B}) receptor as a test agent; and (b) measuring the ability of the selected test agent to modulate drug reward in an animal model. In particular embodiments of the in vivo screening method, a CCK_{B} receptor antagonist is selected as a test agent. In vivo screening can be carried out, in one embodiment, using an animal model that tests expression of conditioned place preference.

Another aspect of the invention is a method of assessing a subject's risk for maladaptive substance use. The method entails determining the level of cholecystokinin-B (CCK_{B}) receptor polypeptides, polynucleotides, or function in a biological sample from the subject, wherein risk for maladaptive substance use is directly correlated with the level. This method can additionally entail administering an inhibitor of a CCK_{B} receptor to a subject determined to have a high risk for maladaptive substance use.

The invention also provides kits. One kit according to the invention includes: (a) an inhibitor of a cholecystokinin-B (CCK_{B}) receptor in a pharmaceutically acceptable carrier; (b) instructions for carrying out a method of the invention for mitigating a symptom of maladaptive substance use. Another kit according to the invention is a diagnostic kit including: (a) a component that specifically binds to a cholecystokinin-B (CCK_{B}) receptor polypeptide or polynucleotide; and (b) instructions for carrying out the method of the invention for assessing a subject's risk for maladaptive substance use.

### BRIEF DESCRIPTION OF THE DRAWINGS

FIG. 1: Anterior (A) and posterior (B) NAcc injection sites. Coordinates in the upper right refer to distance from bregma. CPu, caudate putamen; aca, anterior commisure; AcbS, accumbens shell; AcbC, accumbens core; LV, lateral ventricle. The diameter of each circle indicates the extent of gliosis at each injection site. N=46. Injection sites were marked for each animal in every section on which they were visible.

FIG. 2: The effects of the CCK-B antagonist L-365, 260 (1 mg/kg) on the expression of morphine conditioned place preference following systemic administration. L-365, 260 attenuates the expression of morphine CPP when animals are tested immediately after injection (p=0.006), 24 hours after injection (p=0.0009) and 72 hours after injection (p=0.004).

FIG. 3: (A) L-365, 260 (1 ng) microinjected into the anterior NAcc attenuates the expression of morphine CPP (p=0.002) and L-365, 260 microinjected into the posterior NAcc potentiates the expression of morphine CPP (p=0.021). Both effects are reversed by co-injection of either CCK-4 (10 ng) or CCK-8 (10 ng; B,C), neither of which has an effect on the expression of morphine CPP when injected independently (D, E).

FIG. 4: The specific CCK_{A} antagonist lorglumide (15 ng) has no effect on expression of morphine CPP when microinjected into either the anterior or posterior NAcc.

FIG. 5: When microinjected independently into either the anterior or posterior NAcc, raclopride (4 μg) has no effect on morphine CPP in either the anterior or posterior NAcc (A) but does reverse the effects of L-365, 260 in both locations (B). SCH-23390 (2 μg) potentiates morphine CPP when microinjected independently into the posterior (p=0.049) but not the anterior NAcc (C). Average number of room entries (p=0.007) and beam breaks (p=0.003) following posterior SCH-23390 microinjections (D).

FIG. 6A-D: Rats (n=18) were administered 3 daily doses of systemic L-365 (1 mg/kg) prior to a one-hour ethanol (10%) access session (A). On the second day of L-365, 260 administration, animals were administered 0.8 mA of shock (0.5 seconds every 40 seconds for a period of 15 minutes) in ethanol access chambers directly prior to the onset of their one-hour access session. Animals receiving L-365, 260 consumed significantly less ethanol than saline controls on the two days following shock (B,C). These data can also be seen as cumulative curves charting total ethanol consumption for ethanol and saline control animals (D). This was a randomized cross over design with each animal serving as its own control.

FIG. 7: L-365, 260 had no effect on ethanol consumption when shock was administered during extinction (n=12). Animals were administered 3 daily doses of systemic L-365, 260 after ethanol consumption was extinguished. On the second day of L-365, 260 administration, animals were administered 0.8 mA of shock (0.5 seconds every 40 seconds for a period of 15 minutes) in the ethanol access chambers directly prior to the onset of their one-hour extinction session. This was a randomized cross over design with each animal serving as its own control.

FIG. 8: L-365 had no effect on ethanol reinstatement when shock was administered on the first day of re-initiation of ethanol access (n=12). Animals were administered 3 daily doses of systemic L-365, 260 after ethanol consumption was extinguished. On the first day of L-365, 260 administration, animals were administered 0.8 mA of shock (0.5 seconds every 40 seconds for a period of 15 minutes) in the ethanol access chambers directly prior to the onset of their one-hour reinstatement session. This was a randomized cross over design with each animal serving as its own control.

### DETAILED DESCRIPTION

The present invention relates to the discovery that modulators of the cholecystokinin-B (CCK_{B}) receptor modulate symptoms of maladaptive substance use. Accordingly, the invention provides a method of mitigating a symptom of maladaptive substance use in a subject, as well as a pharmaceutical composition useful in treating maladaptive substance use. The invention also includes various screening methods based on assaying test agents for their ability to bind to, agonize, and/or antagonize CCK_{B} receptors. Another aspect of the invention is a diagnostic method for assessing a subject's risk for maladaptive substance use. These methods are of particular interest with respect to common drugs of abuse, such as opioids, psychostimulants, cannabinoids, empathogens, dissociative drugs, and ethanol.

Definitions

Terms used in the claims and specification are defined as set forth below unless otherwise specified.

The term “receptor” refers to a molecule or complex of molecules, typically (although not necessarily) a protein(s), that is specifically bound by one or more particular ligands. The receptor is said to be a receptor for such ligand(s). Ligand-receptor binding, in many instances, induces one or more biological responses.

The following terms encompass polypeptides that are identified in Genbank by the following designations, as well as polypeptides that are at least about 70% identical to polypeptides identified in Genbank by these designations: cholecystokinin (CCK), cholecystokinin-A (CCK_{A}) receptor, cholecystokinin-B (CCK_{B}) receptor, and mu opioid receptor. In alternative embodiments, these terms encompass polypeptides identified in Genbank by these designations and sharing at least about 80, 90, 95, 96, 97, 98, or 99% identity.

A “modulator” of a receptor (e.g., the CCK_{B} receptor) is either an inhibitor or an enhancer of receptor action.

A “non-selective” modulator of a particular receptor or receptor subtype (e.g., a CCK_{B} receptor) is an agent that modulates other receptors and/or other receptor subtypes at the concentrations typically employed for modulation of the particular receptor or receptor subtype.

A “selective” modulator of a particular receptor or receptor subtype significantly modulates the particular receptor or receptor subtype at a concentration at which other receptors and/or receptor subtypes are not significantly modulated. Thus, a modulator can be selective for, e.g., a CCK receptor or can be selective for a CCK receptor subtype, such as, for example, the CCK_{B} receptor.

A modulator “acts directly on” a receptor or its ligand when the modulator binds to the receptor or ligand, respectively.

A modulator “acts indirectly on” a receptor or its ligand when the modulator binds to a molecule other than the receptor or ligand, which binding results in modulation of receptor or ligand function, respectively.

An “inhibitor” or “antagonist” of a receptor is an agent that reduces, by any mechanism, any receptor action, as compared to that observed in the absence (or presence of a smaller amount) of the agent. An inhibitor of a receptor can affect: (1) the expression, mRNA stability, protein trafficking, modification (e.g., phosphorylation), or degradation of a receptor or one or more of its subunits or of the ligand for the receptor, or (2) one or more of the normal functions of the receptor. An inhibitor of a receptor can be non-selective or selective. Preferred inhibitors (antagonists) are generally small molecules that act directly on, and are selective for, the target receptor.

An “enhancer” or “agonist” is an agent that increases, by any mechanism, any receptor action, as compared to that observed in the absence (or presence of a smaller amount) of the agent. An enhancer of a receptor can affect: (1) the expression, mRNA stability, protein trafficking, modification (e.g., phosphorylation), or degradation of a receptor or one or more of its subunits or of the ligand for the receptor, or (2) one or more of the normal functions of the receptor. An enhancer of a receptor can be non-selective or selective. Preferred enhancers (agonists) are generally small molecules that act directly on, and are selective for, the target receptor.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for He; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

A “radioligand binding assay” is an assay in which a biological sample (e.g., cell, cell lysate, tissue, etc.) containing a receptor is contacted with a radioactively labeled ligand for the receptor under conditions suitable for specific binding between the receptor and ligand, unbound ligand is removed, and receptor binding is determined by detecting bound radioactivity.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

The phrases “an effective amount” and “an amount sufficient to” refer to amounts of a biologically active agent that produce an intended biological activity.

The term “co-administer,” when used in reference to the administration of CCK_{B} and opioid receptor antagonists indicates that the antagonists are administered so that there is at least some chronological overlap in their physiological activity on the organism. Thus the CCK_{B} receptor antagonist can be administered simultaneously and/or sequentially with the opioid receptor antagonist. In sequential administration, there may even be some substantial delay (e.g., minutes or even hours or days) before administration of the second agent as long as the first administered agent is exerting some physiological effect on the organism when the second administered agent is administered or becomes active in the organism.

The term “maladaptive substance use” refers to the use of any substance that results in adverse consequences for the user that outweigh any benefits derived from the substance. Substances that are use in a maladaptive manner are generally consumed or administered (usually self-administered) to the body, by any route of administration, to produce an effect on the body that the user generally experiences as pleasurable. The substance can be a single substance (cocaine, for example) or a type of substance (e.g., food, in general). The adverse consequences can include, for example, adverse effects on health, the ability to care for oneself, the ability to form and maintain human relationships, and/or the ability to work. The adverse consequences are generally significant enough that the user would like to control, reduce, or end substance use or, alternatively, the user's family members and/or friends would like to see the user control, reduce, or end substance use. Maladaptive substance use includes uncontrollable craving for the substance; substance dependence, including psychological and/or physical dependence; and maladaptive substance use; as well as any of the individual symptoms of substance dependence and/or abuse listed below.

A “symptom of maladaptive substance use” includes any symptom arising from maladaptive substance use. Thus, a symptom of maladaptive substance use arises from the previous, and/or ongoing, use of a substance. Examples include, but are not limited to, elevated drug reward, incentive salience for the drug, drug craving, drug seeking, and drug consumption, as compared to that in a normal population (i.e., one that is not using the substance in a maladaptive manner), as well as drug reinstatement, stress-induced reinstatement, and drug relapse or any of the individual symptoms of substance dependence and/or abuse listed below.

“Substance dependence” includes a maladaptive pattern of substance use, leading to clinically significant impairment or distress, as manifested by three (or more) of the following symptoms, occurring at any time in the same 12-month period:

(1) Tolerance, as defined by either of the following: (a) a need for markedly increased amounts of the substance to achieve intoxication or desired effect, or (b) markedly diminished effect with continued use of the same amount of the substance;

(2) Withdrawal, as manifested by either of the following: (a) the characteristic withdrawal syndrome for the substance, or (b) the same (or closely related) substance is taken to relieve or avoid withdrawal symptoms;

(3) The substance is often taken in larger amounts or over a longer period than was intended;

(4) There is a persistent desire or unsuccessful efforts to cut down or control substance use;

(5) A great deal of time is spent in activities necessary to obtain the substance (e.g., visiting multiple doctors or driving long distances), use the substance (e.g., chain-smoking), or recover from its effects;

(6) Important social, occupational, or recreational activities are given up or reduced because of substance use; and

(7) The substance use is continued despite knowledge of having a persistent or recurrent physical or psychological problem that is likely to have been caused or exacerbated by the substance (e.g., current cocaine use despite recognition of cocaine-induced depression, or continued drinking despite recognition that an ulcer was made worse by alcohol consumption). (See American Psychiatric Association, Diagnostic Criteria for DSM-IV, Washington D.C., APA, 1994.)

A person is “dependent upon a substance” if such person is determined by a licensed physician or other appropriate accredited medical personnel to meet the criteria for substance dependence with respect to such substance.

“Substance abuse” includes a maladaptive pattern of substance use leading to clinically significant impairment or distress, as manifested by one (or more) of the following, occurring within a 12-month period:

(1) recurrent substance use resulting in a failure to fulfill major role obligations at work, school, or home (e.g., repeated absences or poor work performance related to substance use; substance-related absences, suspensions, or expulsions from school; neglect of children or household);

(2) recurrent substance use in situations in which it is physically hazardous (e.g., driving an automobile or operating a machine when impaired by substance use);

(3) recurrent substance-related legal problems (e.g., arrests for substance-related disorderly conduct); and

(4) continued substance use despite having persistent or recurrent social or interpersonal problems caused or exacerbated by the effects of the substance (e.g., arguments with spouse about consequences of intoxication, physical fights). (See American Psychiatric Association, Diagnostic Criteria for DSM-IV, Washington D.C., APA, 1994.)

A person is “an abuser of a substance” or “abusive of a substance” if such person is determined by a licensed physician or other appropriate accredited medical personnel to meet the criteria for substance abuse with respect to such substance.

The terms drug reward, incentive salience for the drug, drug craving, drug seeking, drug consumption, drug reinstatement, and drug relapse refer to “drugs” because these concepts have generally been used in the drug dependence/abuse context. However, it should be understood that these terms, as used herein, also encompass reward, incentive salience, craving, seeking and consumption of any substance that is used in a maladaptive manner.

The term “drug reward” refers to the tendency of a drug or substance to cause pleasurable effects which induce a subject to alter their behavior to obtain more of the drug or substance.

The phrase “incentive salience for the drug” refers to a particular form of motivation to consume a previously experienced drug or substance that results from a hypersensitive neural state thought to be mediated by dopaminergic systems.

The term “drug craving” refers to the desire to experience the effects of a previously experienced drug or substance or to ameliorate the negative symptoms of drug or substance withdrawal by taking more of a previously experienced drug or substance.

The term “drug seeking” refers to behavior aimed at obtaining a drug or substance, even in the face of negative health and social consequences. Drug seeking is often uncontrollable and compulsive.

“Drug consumption” refers to the amount of drug or substance consumed by a subject over a selected period of time.

[*JENNY, PLEASE REVIEW THE FOLLOWING DEFINITIONS AND REVISE, IF NECESSARY.]

The term “drug reinstatement” refers to the reinstatement of any behavior related to maladaptive substance use that a subject exhibits while using a drug or other substance and that extinguishes during a period of non-use of the drug or substance. Drug reinstatement can be assessed, for example, by measuring any behavior associated with self-administration of a drug.

The term “stress-induced reinstatement” refers to the stress-induced reinstatement of any behavior related to maladaptive substance use that a subject exhibits while using a drug or other substance and that extinguishes during a period of non-use of the drug or substance.

The term “drug relapse” refers to a subject's return to maladaptive substance use after a period of non-use or a period during which the substance was not used in a maladaptive manner.

A “drug of abuse” includes any substance, the excessive consumption or administration of which can result in a diagnosis of substance dependence or abuse as defined herein or as defined by the current DSM Criteria promulgated by the American Psychiatric Association or equivalent criteria. Drugs of abuse include, without limitation, an opioid, a psychostimulant, a cannabinoid, an empathogen, a dissociative drug, and ethanol. Thus, for example, heroin, cocaine, methamphetamines, cannabis, 3-4 methylenedioxy-methamphetamine (MDMA), barbiturates, phencyclidine (PCP), ketamine, and ethanol are all drugs of abuse, as defined herein.

A “test agent” is any agent that can be screened in the prescreening or screening assays of the invention. The test agent can be any suitable composition, including a small molecule, peptide, or polypeptide.

An agent is said to “modulate” a symptom of maladaptive substance use if the agent inhibits (i.e., reduces or prevents) or enhances (i.e., increases) the symptom.

An agent is said to “mitigate” a symptom of maladaptive substance use if the agent inhibits (i.e., reduces or prevents) the symptom.

The term “therapy,” as used herein, encompasses the treatment of an existing condition as well as preventative treatment (i.e., prophylaxis). Accordingly, “therapeutic” effects and applications include prophylactic effects and applications, respectively.

A used herein, the term “high risk” refers to an elevated risk as compared to that of an appropriate matched (e.g., for age, sex, etc.) control population.

Method of Mitigating a Symptom of Maladaptive Substance Use

A. In General

The invention provides a method of mitigating a symptom of maladaptive substance use. The method entails inhibiting a CCK_{B} receptor in a subject, whereby the symptom of maladaptive substance use is reduced or prevented. Generally, the method is carried out by systemically administering an effective amount of a CCK_{B} receptor inhibitor to a subject. The invention encompasses methods applicable to the maladaptive use of any substance, including any substance that is used maladaptively that is described individually herein, any grouping of individually described substances, any generic group of substances described herein, and any generic group of substances described herein, with the proviso that one or more of the individually described substances is excluded from the generic group. In particular, the method is useful for addressing undesirable effects or behaviors associated with a variety of drugs of abuse, as well as those associated with other substances, such as food, particular types of food (e.g., sugar, caffeine), and nicotine. In one embodiment, the method is employed to treat maladaptive use of any substance that is used maladaptively, except food. In another embodiment, the method is employed to treat maladaptive use of any substance that it used maladaptively except food and nicotine. In yet another embodiment, the method is employed to treat maladaptive use of a drug of abuse.

In particular embodiments, the method is used to reduce or prevent symptoms associated with drugs such as opioids, psychostimulants, cannabinoids, empathogens, ethanol, and the like. Exemplary opioids include morphine, codeine, heroin, butorphanol, hydrocodone, hydromorphone, levorphanol, meperidine, nalbuphine, oxycodone, fentanyl, methadone, propoxyphene, remifentanil, sufentanil, and pentazocine. Psychostimulants include drugs that stimulate the central nervous system, such as, for example, amphetamine, cocaine, methamphetamine, methylphenidate (ritalin), and methylene dioxy-methamphetamine (MDMA). Exemplary cannabinioids include tetrahydrocannabinol (THC), dronabinol, and arachidonylethanolamide (anandamide, AEA). Empathogens include phenethylamines, such as, for example, MDMA, 3,4-methylenedioxy amphetamine (MDA), 3,4-methylenedioxy-N-ethylamphetamine (MDEA), 2,5-Dimethoxy-4-iodo-phenethylamine or 1-(2,5-dimethoxy-4-iodophenyl)-2-aminoethane (2C-I), 2,5-dimethoxy-4-bromo-phenethylamine (2C-B), and N-methyl-1-(3,4-methylenedioxyphenyl)-2-butanamine. Dissociative drugs include PCP and ketamine.

Examples of symptoms of maladaptive substance use that can be mitigated according to the method of the invention include elevated: drug reward, incentive salience for a drug, drug craving, drug seeking, drug consumption, drug reinstatement, stress-induced reinstatement, and drug relapse.

The subject of the method can be any individual that has CCK_{B} receptors. Examples of suitable subjects include research animals, such as mice, rats, guinea pigs, rabbits, cats, dogs, as well as monkeys and other primates, and humans. The subject can be an individual who is regularly, or intermittently, using a substance in a maladaptive manner or an individual who is at risk for such use. In particular embodiments, the subject uncontrollably craves, is physically or psychologically dependent upon, or an abuser of, a drug of abuse. The method finds particular application in treating subjects in whom craving, physical or psychological drug dependence, and/or abuse has recently been identified, (e.g., as part of a drug rehabilitation program) or who have been found through genetic testing to be at risk.

The method of the invention entails inhibiting the CCK_{B} receptor to a degree sufficient to reduce or prevent one or more symptom(s) of maladaptive substance use. In various embodiments, the CCK_{B} receptor is inhibited by at least about 10, 20, 30, 40, 50, 60, 70, 80, 90, and 95 percent, as determined by any suitable measure of CCK_{B} receptor inhibition (such as, for example, any of the assays described herein).

Any kind of CCK_{B} receptor inhibitor that is tolerated by the subject can be employed in the method of the invention. Thus, the inhibitor can be a polypeptide (such as, e.g., an anti-CCK_{B} receptor antibody), a polynucleotide (e.g., one that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit CCK_{B} receptors.

Inhibition of the CCK_{B} receptor can be achieved by any available means, e.g., inhibition of: (1) the expression, mRNA stability, protein trafficking, modification (e.g., phosphorylation), or degradation of a CCK_{B} receptor, or (2) one or more of the normal functions of a CCK_{B} receptor such as ligand binding, G-protein interaction, internalization, dimerization, decoupling, upregulation, and channel gating.

In one embodiment, CCK_{B} receptor inhibition is achieved by reducing the level of CCK_{B} receptors in a target tissue having such receptors. CCK_{B} receptors are expressed in neurons of the central nervous system. Thus, the method of the invention can reduce CCK_{B} receptor levels in areas of the brain such as the substantia nigra pars compacta, the ventral tegmental area, the prefrontal cortex, and/or the nucleus accumbens (NAcc; which includes CCK-containing projections from the VTA and the prefrontal cortex). CCK_{B} receptor levels can be reduced using, e.g., antisense or RNA interference (RNAᵢ) techniques.

In preferred embodiments, the CCK_{B} receptor inhibitor can be a CCK receptor antagonist, which can be, e.g., a peptide or a small molecule. A large number of small-molecule CCK antagonists have been described, including A-65186 (N-3-quinolinoyl-D-Glu-N,N-dipentylamide); C1988, L-364,718 (MK329=devazepide); compounds isolated from Streptomyces (tetronothiodin, virginiamycin analogues), ureido-acetamide analogues (RP 69758, RP 72540, RP 73870), benzodiazepine analogues (L-365,260, L-368,935, L-740,093, YM022), pyrazolidimine analogues (LY 262,691), and glutamic acid analogues (CR2194). See Dunlop, J., Gen Pharmacol. (1998) 4:519-24; Jensen, R. T., Yale J Biol Med. (1996) 69:245-59; Xu, X. J. et al., Br J Pharmacol. (1992) 105:591-6); U.S. Pat. No. 4,696,925, issued Sep. 29, 1987 to Houck, et al.; U.S. Pat. No. 4,814,463, issued Mar. 21, 1989 to Kim; U.S. Pat. No. 5,686,449, issued Nov. 11, 1977 to Tranquillini, et al.

The CCK_{B} receptor inhibitor can be non-selective or selective for CCK_{B} receptors. Preferred embodiments employ a selective CCK_{B} receptor antagonist, such as, for example, C1988, compounds isolated from Streptomyces (tetronothiodin, virginiamycin analogues), ureido-acetamide analogues (RP 69758, RP 72540, RP 73870), benzodiazepine analogues (L-365,260, L-368,935, L-740,093, YM022), pyrazolidimine analogues (LY 262,691), and glutamic acid analogues (CR2194). See Dunlop, J., Gen Pharmacol. (1998) 4:519-24; Jensen, R. T., Yale J Biol Med. (1996) 69:245-59; Xu, X. J. et al., Br J Pharmacol. (1992) 105:591-6). Additional CCK_{B} receptor-selective antagonists are described in U.S. Pat. No. 5,686,449, issued Nov. 11, 1977 to Tranquillini, et al.

In a particular embodiment of the method, a CCK_{B} receptor inhibitor is co-administered with an inhibitor of an opioid receptor. This embodiment is useful, for example, in treating former opioid abusers to reduce the risk of relapse. More specifically, an opioid receptor inhibitor can be administered in conjunction with a CCK_{B} receptor inhibitor to prevent opioid overdose in the event that the subject relapses. In the absence of the opioid receptor inhibitor, the subject may be at increased risk for overdosing because the CCK_{B} receptor inhibitor will block a portion of the opioid-induced, and opioid-specific, tolerance, potentiating the physiological side-effects of the opioid and thereby increasing the possibility of overdose. In this embodiment, the amount of the opioid receptor inhibitor administered is sufficient to reduce the risk of drug overdose in the subject.

Any kind of opioid receptor inhibitor that is tolerated by the subject can be employed in the method of the invention. Thus, the inhibitor can be a polypeptide (such as, e.g., an anti-opioid receptor antibody), a polynucleotide (e.g., one that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit opioid receptors.

Inhibition of opioid receptor can be achieved by any available means, e.g., inhibition of: (1) the expression, mRNA stability, protein trafficking, modification (e.g., phosphorylation), or degradation of a opioid receptor, or (2) one or more of the normal functions of a opioid receptor, such as ligand binding, G-protein interaction, internalization, dimerization, decoupling, upregulation, and channel gating.

In one embodiment, opioid receptor inhibition is achieved by reducing the level of opioid receptors in a target tissue having such receptors. Opioid receptors are expressed in neurons of the central nervous system, as well as in peripheral tissues. Thus, the method of the invention can reduce opioid receptor levels in areas of the brain such as the nucleus accumbens (NAcc), ventral tegmental area (VTA), amygdala, prefrontal cortex, bed nucleus of the stria terminalis (BNST), and/or hypothalamus. Opioid receptor levels can be reduced using, e.g., antisense or RNA interference (RNAᵢ) techniques.

In preferred embodiments, the opioid receptor inhibitor can be an opioid receptor antagonist, which can be, e.g., a peptide or a small molecule. The opioid receptor inhibitor can be non-selective or selective for particular opioid receptors. Preferred embodiments employ a selective mu opioid receptor antagonist, such as, for example, naltrexone, naloxone, CTAP, or CTOP.

Agents that act via the CCK_{B} and opioid receptors can be co-administered by simultaneous administration or sequential administration. In the case of sequential administration, the first administered agent must be exerting some physiological effect on the organism when the second administered agent is administered or becomes active in the organism.

B. Compositions

For research and therapeutic applications, a CCK_{B} receptor inhibitor is generally formulated to deliver inhibitor to a target site in an amount sufficient to inhibit CCK_{B} receptors at that site. An opioid receptor inhibitor can, optionally, be included in the formulation to deliver an amout of opioid receptor inhibitor sufficient to inhibit opioid receptors at the target site.

Inhibitor compositions of the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Preferred embodiments include sustained-release pharmaceutical compositions. An exemplary sustained-release composition has a semipermeable matrix of a solid hydrophobic polymer to which the inhibitor is attached or in which the inhibitor is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are typically in the form of shaped articles, such as films, or microcapsules.

Where the inhibitor is a polypeptide, exemplary sustained release compositions include the polypeptide attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” protein retains the desired function(s).

In another embodiment, a sustained-release composition includes a liposomally entrapped inhibitor. Liposomes are small vesicles composed of various types of lipids, phospholipids, and/or surfactants. These components are typically arranged in a bilayer formation, similar to the lipid arrangement of biological membranes. Liposomes containing CCK_{B} (and, optionally opioid) receptor inhibitors are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide inhibitor or a polynucleotide encoding a polypeptide inhibitor of CCK_{B} (and, optionally opioid) receptors. Such compositions optionally include other components, as for example, a storage solution, such as a suitable buffer, e.g., a physiological buffer. In a preferred embodiment, the composition is a pharmaceutical composition and the other component is a pharmaceutically acceptable carrier, as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

C. Administration

Pharmaceutical compositions according to the invention are generally administered systemically. Methods for systemic administration do not differ from known methods for administering small-molecule drugs or therapeutic polypeptides, peptides, or polynucleotides them. Suitable routes of administration include, for example, topical, intravenous, intraperitoneal, intracerebral, intraventricular, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

In certain embodiments, the compositions are delivered through the skin using a conventional transdermal drug delivery system, i.e., a transdermal “patch” wherein the composition is typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of a selected composition that is ultimately available for delivery to the surface of the skin. Thus, for example, the reservoir may include the composition in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the reservoir and skin contact adhesive are present as separate and distinct layers, with the adhesive underlying the reservoir which, in this case, may be either a polymeric matrix as described above or a liquid or hydrogel reservoir, or it may take some other form. The backing layer in these laminates, which serves as the upper surface of the device, preferably functions as a primary structural element of the patch and provides the device with much of its flexibility. The material selected for the backing layer is preferably substantially impermeable to the selected composition and any other materials that are present.

Transdermal patches according to the invention can include a rate-limiting patch membrane. The size of the patch and or the rate-limiting membrane can be chosen to deliver the transdermal flux rates desired. A release liner, such as a polyester release liner, can also be provided to cover the adhesive layer prior to application of the patch to the skin as is conventional in the art. This patch assembly can be packaged in an aluminum foil or other suitable pouch, again, as is conventional in the art.

In other embodiments, the compositions of the invention are administered in implantable depot formulations. A wide variety of approaches to designing depot formulations that provide sustained release of an active agent are known and are suitable for use in the invention. Generally, the components of such formulations are biocompatible and may be biodegradable. Biocompatible polymeric materials have been used extensively in therapeutic drug delivery and medical implant applications to effect a localized and sustained release. See Leong et al., “Polymeric Controlled Drug Delivery”, Advanced Drug Delivery Rev., 1: 199-233 (1987); Langer, “New Methods of Drug Delivery”, Science, 249:1527-33 (1990); Chien et al., Novel Drug Delivery Systems (1982). Such delivery systems offer the potential of enhanced therapeutic efficacy and reduced overall toxicity.

If an implant is intended for use as a drug delivery or other controlled-release system, using a biodegradable polymeric carrier is one effective means to deliver the therapeutic agent locally and in a controlled fashion, see Langer et al., “Chemical and Physical Structures of Polymers as Carriers for Controlled Release of Bioactive Agents”, J. Macro. Science, Rev. Macro. Chem. Phys., C23(1), 61-126 (1983). As a result, less total drug is required, and toxic side effects can be minimized. Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists”, Controlled Release of Bioactive Materials, 19-44 (Richard Baker ed., 1980); poly(amino acids) and pseudo-poly(amino acids) (Pulapura et al. “Trends in the Development of Bioresorbable Polymers for Medical Applications”, J. Biomaterials Appl., 6:1, 216-50 (1992); polyurethanes (Bruin et al., “Biodegradable Lysine Diisocyanate-based Poly(Glycolide-co-.epsilon. Caprolactone)-Urethane Network in Artificial Skin”, Biomaterials, 11:4, 291-95 (1990); polyorthoesters (Heller et al., “Release of Norethindrone from Poly(Ortho Esters)”, Polymer Engineering Sci., 21:11, 727-31 (1981); and polyanhydrides (Leong et al., “Polyanhydrides for Controlled Release of Bioactive Agents”, Biomaterials 7:5, 364-71 (1986).

Thus, for example, a CCK_{B} receptor inhibitor composition (optionally containing an opioid receptor inhibitor) can be incorporated into a biocompatible polymeric composition and formed into the desired shape outside the body. This solid implant is then typically inserted into the body of the subject through an incision. Alternatively, small discrete particles composed of these polymeric compositions can be injected into the body, e.g., using a syringe. In an exemplary embodiment, a CCK_{B} receptor inhibitor composition (optionally containing an opioid receptor inhibitor) can be encapsulated in microspheres of poly (D,L-lactide) polymer suspended in a diluent of water, mannitol, carboxymethyl-cellulose, and polysorbate 80. The polylactide polymer is gradually metabolized to carbon dioxide and water, releasing the CCK_{B} receptor inhibitor into the system.

In yet another approach, depot formulations can be injected via syringe as a liquid polymeric composition. Liquid polymeric compositions useful for biodegradable controlled release drug delivery systems are described, e.g., in U.S. Pat. Nos. 4,938,763; 5,702,716; 5,744,153; 5,990,194; and 5,324,519. After injection in a liquid state or, alternatively, as a solution, the composition coagulates into a solid.

One type of polymeric composition suitable for this application includes a nonreactive thermoplastic polymer or copolymer dissolved in a body fluid-dispersible solvent. This polymeric solution is placed into the body where the polymer congeals or precipitates and solidifies upon the dissipation or diffusion of the solvent into the surrounding body tissues. See, e.g., Dunn et al., U.S. Pat. Nos. 5,278,201; 5,278,202; and 5,340,849 (disclosing a thermoplastic drug delivery system in which a solid, linear-chain, biodegradable polymer or copolymer is dissolved in a solvent to form a liquid solution).

A CCK_{B} receptor inhibitor composition (optionally containing an opioid receptor inhibitor) can also be adsorbed onto a membrane, such as a silastic membrane, which can be implanted, as described in International Publication No. WO 91/04014.

D. Dose

The dose of inhibitor is sufficient to inhibit the target receptor, preferably without significant toxicity. In particular in vivo embodiments, the amount of the CCK_{B} receptor inhibitor is sufficient to mitigate a symptom of maladaptive substance use in a subject. In variations of this embodiment in which an opioid receptor inhibitor is co-administered with the CCK_{B} receptor inhibitor, the amount of the opioid receptor inhibitor is sufficient to reduce the risk of drug overdose in the subject. For in vivo applications, the dose of inhibitor depends, for example, upon the therapeutic objectives, the route of administration, and the condition of the subject. Accordingly, it is necessary for the clinician to titer the dosage and modify the route of administration as required to obtain the optimal therapeutic effect. Generally, the clinician begins with a low dose and increases the dosage until the desired therapeutic effect is achieved. Starting doses for a given inhibitor can be extrapolated from in vitro data.

For example, the CCK_{B} receptor inhibitor L-365,260 can be administered in an amount approximately equivalent to an oral dose of between about 10 to about 100 mg/day, more typically between about 25 to about 75 mg/day, and most typically about 50 mg/day. The mu opioid receptor inhibitor naltrexone can be administered in an amount approximately equivalent to an oral dose of about 1 mg/kg to about 3 mg/kg per day, depending on the weight of the subject. Thus, a typical oral dose of naltrexone is in the range of about 50 mg to about 200 mg per day.

Methods of Screening for Agents that Modulate a Symptom of Maladaptive Substance Use

The role of CCK_{B} receptors in mediating the reward associated with substance use makes this receptor an attractive target for agents that modulate one or more symptoms of maladaptive substance use. Accordingly, the invention provides prescreening and screening methods aimed at identifying such agents. Test agents can be prescreened, for example, based on binding to CCK_{B} receptors or on binding to polynucleotides encoding CCK_{B} receptors. Screening methods of the invention can be carried out by: contacting a test agent with a CCK_{B} receptor; determining whether the test agent acts as an agonist or an antagonist of the receptor; and if so, selecting the test agent as a potential modulator of maladaptive substance use in a subject. For example, test agents can be screened for effects on the levels of CCK_{B} receptors or polynucleotides encoding them (e.g., CCK_{B} mRNA) or for effects on CCK_{B} receptor function.

The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof.

A. Prescreening Based on Binding to CCK_{B} Receptors

The invention provides a prescreening method based on assaying test agents for specific binding to a CCK_{B} receptor. Agents that specifically bind to CCK_{B} receptors have the potential to modulate receptor function and thereby modulate one or more symptoms of maladaptive substance use.

In one embodiment, therefore, a prescreening method of the invention entails contacting a test agent with a CCK_{B} receptor. Specific binding of the test agent to the receptor is then determined. If specific binding is detected, the test agent is selected as a potential modulator of a symptom of maladaptive substance use.

In a variation of this embodiment, the test agent can be screened to determine relative selectivity for the CCK_{B} receptor versus the cholecystokinin-A (CCK_{A}) receptor. In this instance, the test agent is also contacted with a CCK_{A} receptor, and specific binding of the test agent to the CCK_{A} receptor is determined. If test agent preferentially binds to the CCK_{B} receptor over the CCK_{A} receptor, the test agent is selected as a potential modulator of a symptom of maladaptive substance use.

Such prescreening is generally most conveniently accomplished with a simple in vitro binding assay. Means of assaying for specific binding of a test agent to a polypeptide are well known to those of skill in the art. In preferred binding assays, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various assay formats are discussed in greater detail below.

B. Prescreening Based on Binding to Polynucleotides Encoding CCK_{B} Receptor

The invention also provides a prescreening method based on screening test agents for specific binding to a polynucleotide encoding a CCK_{B} receptor. Agents that specifically bind to such polynucleotides have the potential to modulate the expression of the encoded CCK_{B} receptor, and thereby modulate one or more symptoms of maladaptive substance use.

In one embodiment, therefore, a prescreening method of the invention entails contacting a test agent with a polynucleotide encoding a CCK_{B} receptor subunit. Specific binding of the test agent to the polynucleotide is then determined. If specific binding is detected, the test agent is selected as a potential modulator of a symptom of maladaptive substance use.

Such prescreening is generally most conveniently accomplished with a simple in vitro binding assay, which are well known to those of skill in the art. In preferred binding assays, the polynucleotide is immobilized and exposed to a test agent (which can be labeled), or alternatively, the test agent(s) are immobilized and exposed to the polynucleotide (which can be labeled). The immobilized species is then washed to remove any unbound material and the bound material is detected. To prescreen large numbers of test agents, high throughput assays are generally preferred. Various assay formats are discussed in greater detail below.

C. Screening Based on Levels of CCK_{B} Receptors or Receptor Polynucleotides

Test agents, including, for example, those identified in a prescreening assay of the invention can also be screened to determine whether the test agent affects the levels of CCK_{B} receptors or receptor polynucleotides (e.g., mRNA). Agents that reduce these levels can potentially reduce one or more symptoms of maladaptive substance use. Conversely, agents that increase these levels can potentially enhance such symptom(s).

Accordingly, the invention provides a method of screening for an agent that modulates a symptom of maladaptive substance use in which a test agent is contacted with a cell that expresses a CCK_{B} receptor in the absence of test agent. Preferably, the method is carried out using an in vitro assay. In such assays, the test agent can be contacted with a cell in culture or present in a tissue. Alternatively, the test agent can be contacted with a cell lysate or fraction thereof. The level of CCK_{B} receptors or CCK_{B} receptor polynucleotides (e.g., mRNA) is determined in the presence and absence (or presence of a lower amount) of test agent to identify any test agents that alter the level. If the level is altered, the test agent is selected as a potential modulator of a symptom of maladaptive substance use. In a preferred embodiment, an agent that reduces the CCK_{B} receptor or receptor polynucleotide level is selected as a potential inhibitor of one or more symptoms of maladaptive substance use.

In particular embodiments, the screening method entails screening for agents that selectively affect the CCK_{B} receptor or receptor polynucleotide level. In this embodiment, the level of CCK_{A} receptors or polynucleotides (e.g., mRNA) is determined in the presence and absence (or presence of a lower amount) of test agent. Any agent that preferentially affects CCK_{B} receptor and/or receptor polynucleotide levels over CCK_{A} receptor and/or receptor polynucleotide levels is selected as a potential modulator of maladaptive substance use.

Cells or tissues useful in this screening method include those from any of the species described above in connection with the method of mitigating a symptom of maladaptive substance use. Cells that naturally express a CCK_{B} receptor are typically, although not necessarily, employed in this screening method. Examples include, but are not limited to, SNU-245, SNU-308, SNU-478, SNU-869, SNU-1079, SNU-1196, SNU-213, SNU-324, SNU-410, MIAPaCa-2, and PANC-1. Alternatively, cells that have been engineered to express a CCK_{B} receptor can be used in the method.

1. Sample

As noted above, screening assays are generally carried out in vitro, for example, in cultured cells, in a biological sample (e.g., brain), or fractions thereof. For ease of description, cell cultures, biological samples, and fractions are referred to as “samples” below. The sample is generally derived from an animal (e.g., any of the research animals mentioned above), preferably from a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

2. Polypeptide-Based Assays

CCK_{B} receptors can be detected and quantified by any of a number of methods well known to those of skill in the art. Examples of analytic biochemical methods suitable for detecting CCK_{B} receptors include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one embodiment, CCK_{B} receptors are detected/quantified using a ligand binding assay, such as, for example, a radioligand binding assay. Briefly, a sample from a tissue expressing CCK_{B} receptors is incubated with a suitable ligand under conditions designed to provide a saturating concentration of ligand over the incubation period. After ligand treatment, the sample is assayed for radioligand binding. Any ligand that binds to CCK_{B} receptors can be employed in the assay, although CCK_{B}-selective ligands are preferred. Any of the CCK_{B} receptor inhibitors discussed above can, for example, be labeled and used in this assay. An exemplary, preferred ligand for this purpose is L-365, 260. Binding of this ligand to cells can be assayed as described, for example, in Chang et al., 1989.

In another embodiment, CCK_{B} receptors are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of CCK_{B} receptors in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In a preferred embodiment, CCK_{B} receptors are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case a CCK_{B} receptor). In preferred embodiments, the capture agent is an antibody.

Immunoassays also typically utilize a labeling agent to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the labeling agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled CCK_{B} receptor is added to the sample, and the sample is then contacted with a capture agent. The amount of labeled receptor bound to the antibody is inversely proportional to the concentration of receptor present in the sample.

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In particular embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen, or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Antibodies useful in these immunoassays include polyclonal and monoclonal antibodies.

3. Polynucleotide-Based Assays

Changes in CCK_{B} receptor expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part L Theory and Nucleic Acid Preparation, Elsevier, N.Y. and Tijssen ed.

i. Amplification-Based Assays

In one embodiment, amplification-based assays can be used to detect, and optionally quantify, a polynucleotide encoding the CCK_{B} receptor. In exemplary amplification-based assays, the CCK_{B} receptor mRNA in the sample acts as a template in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of CCK_{B} receptor mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

ii. Hebridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of CCK_{B} receptor mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the CCK_{B} mRNA of interest, and standard methods (such as, e.g., densitometry where the nucleic acid probe is radioactively labeled) can be used to quantify the level of the CCK_{B} mRNA.)

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g., by hand using a pipette) different nucleic acids at different locations on a solid support (e.g., a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a preferred embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can also be carried out using a MicroElectroMechanical System (MEMS), such as the Protiveris' multicantilever array.

iii. Polynucleotide Detection

CCK_{B} polynucleotides can be detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000, 7700, or 7900 (TaqMan®) from Applied Biosystems or the LightCycler® System from Roche.

D. Screening Based on CCK_{B} Receptor Function

The invention also provides a screening method based on determining the effect, if any, of a test agent on the level of a CCK_{B} receptor function. CCK_{B} receptor function can be assayed my measuring any response mediated by CCK_{B} receptors. Agents that reduce CCK_{B} receptor function can potentially reduce one or more symptoms of maladaptive substance use. Conversely, agents that increase CCK_{B} receptor function can potentially enhance such symptom(s).

Accordingly, the invention provides a method of screening for an agent that inhibits or enhances a symptoms of maladaptive substance use in which a test agent is contacted with a cell that expresses a CCK_{B} receptor in the absence of test agent. Preferably, the method is carried out using an in vitro assay. In such assays, the test agent can be contacted with a cell in culture or present in a tissue. The level of CCK_{B} receptor function is determined in the presence and absence (or presence of a lower amount) of test agent to identify any test agents that alter the level. If the level of CCK_{B} receptor function is altered, the test agent is selected as a potential modulator of a symptom of maladaptive substance use. In a preferred embodiment, an agent that reduces CCK_{B} receptor function is selected as a potential inhibitor of one or more symptoms of maladaptive substance use.

Cells or tissues useful for screening based on CCK_{B} receptor function include any of those described above in connection with screening based on levels of CCK_{B} receptors or CCK_{B} receptor polynucleotides.

CCK receptor function can be measured using any assay for any CCK receptor response. Examples of suitable assays include the measurement of: CCK-induced calcium mobilization, as described, e.g., by Williams et al. (1988) PNAS 85:4939-494 and Muller et al. (1997) J Neurophysiol 78:1997-2001; phosphoinoside turnover, as described, e.g., by Jagerschmidt et al. (1995) Mol. Pharm. 48:783-789; excitatory responses in neurons, as described, e.g., in Schutte et al. (1997) J Auton Nerv Syst. 67:51-9.

In particular embodiments, the screening method entails screening for agents that are selective for the CCK_{B} receptor. In such embodiments, the level of CCK_{A} receptor function is determined in the presence and absence (or presence of a lower amount) of test agent. Any agent that preferentially affects CCK_{B} receptor function over CCK_{A} receptor function is selected as a potential modulator of maladaptive substance use. Preferential effects on CCK_{B} function can be determined by assaying the effects of a test agent on a CCK_{B} receptor-mediated response and on a CCK_{A}-receptor mediated response and compared the two. A relatively larger alteration in the CCK_{B} response versus the CCK_{A} response indicates that the test agent preferentially affects CCK_{B} receptor function. The effects of a test agent on the different receptor isoforms can be assessed using cells that express only one isoform, e.g., cells of a given type that do not normally express CCK receptors can be engineered to generate one cell line that expresses CCK_{B} and a second cell line that expresses CCK_{A}. The effects of a test agent on CCK_{B} versus CCK_{A} receptor function can also be assessed in cells that express both receptors using isoform-specific agonists or non-specific agonists in combination with isoform-specific antagonists. Thus, for example, CCK_{A} receptors can be blocked with a CCK_{A} receptor-specific antagonist, allowing a determination as to the effect of a test agent on a response to a non-specific CCK agonist.

E. Test Agent Databases

In a preferred embodiment, generally involving the screening of a large number of test agents, the screening method includes the recordation of any test agent selected in any of the above-described prescreening or screening methods in a database of agents that may modulate a symptom of maladaptive substance use in a subject.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

F. Test Agents Identified by Screening

When a test agent is found to alter the level of CCK_{B} receptors, receptor polynucleotides, or function, a preferred screening method of the invention further includes combining the test agent with a carrier, preferably a pharmaceutically acceptable carrier, such as are described above. Generally, the concentration of test agent is sufficient to alter the level of CCK_{B} receptors, receptor polynucleotides, or function when the composition is contacted with a cell. This concentration will vary, depending on the particular test agent and specific application for which the composition is intended. As one skilled in the art appreciates, the considerations affecting the formulation of a test agent with a carrier are generally the same as described above with respect to methods of mitigating a symptom of maladaptive substance use.

In a preferred embodiment, the test agent is administered to an animal to measure the ability of the selected test agent to modulate a symptom of maladaptive substance use in a subject, as described in greater detail below.

G. Screening For Modulation of a Symptom of Maladaptive Substance Use

The invention also provides a method of screening for an agent that that modulates a symptom of maladaptive substance use in a subject. The method entails selecting a CCK_{B} receptor modulator as a test agent, and measuring the ability of the selected test agent to modulate maladaptive substance use in a subject. Any agent that modulates CCK_{B} receptors and that can be administered to a subject can be employed in the method. Test agents selected through any of the prescreening or screening methods of the invention can be tested for modulation of maladaptive substance use. Alternatively, known CCK_{B} modulators can be employed. In a preferred embodiment, the selected test agent is a CCK_{B} receptor inhibitor.

Test agents can be formulated for administration to a subject as described above for CCK_{B} receptor inhibitors.

The subject of the method can be any individual that has CCK_{B} receptors and in which symptoms of maladaptive substance use can be measured. Examples of suitable subjects include research animals, such as Drosophila melanogaster, mice, rats, guinea pigs, rabbits, cats, dogs, as well as monkeys and other primates, and humans. In preferred embodiments, an animal model established for studying particular drug-related effects or behaviors is employed. In an exemplary, preferred embodiment, the ability of a test agent to modulate drug reward in an animal model is measured. For instance, the animal model can be one that tests the expression of conditioned place preference, as described in Example 1.

The test agent is administered to the subject before, during, and/or after administration of the substance of interest, and the subject is tested or observed to determine whether the test agent modulates a particular symptom of maladaptive substance use. Test agents can be administered by any suitable route, as described above for CCK_{B} receptor inhibitors. Generally, the concentration of test agent is sufficient to alter the level of CCK_{B} receptors, receptor polynucleotides, or function in vivo.

The substance and symptom of maladaptive substance use studied can be any of those described above (e.g., drug reward, incentive salience for the drug, drug craving, drug seeking, drug consumption, drug reinstatement, stress-induced reinstatement, and drug relapse). The substance is administered by any suitable route and in an amount sufficient-to produce the symptom under examination. The symptom is measured and compared with that observed in the absence of test agent and/or in the presence of a lower amount of test agent.

H. Correlation of CCK_{B} Gene Structure with Response to CCK_{B} Receptor Modulators

The invention also provides a method wherein CCK_{B} gene structure is determined in one or more subjects that respond to one or more CCK_{B} receptor modulators. Thus, for example, single nucleotide polymorphisms (SNPs) can be examined to determine whether a particular sequence is correlated with response to one or more CCK_{B} receptor modulators. This information can be used to classify CCK_{B} receptor modulators, e.g., to classify enhancers or inhibitors according to whether they produce a response in a subject with a particular CCK_{B} gene structure. Prior to administering a CCK_{B} receptor inhibitor, for example, in a method of modulating a symptom of maladaptive substance use, CCK_{B} gene structure can be analyzed in the subject to determine the likelihood that the subject will respond to a particular inhibitor and/or select the inhibitor most suitable for treating that subject.

Method of Assessing Risk of Maladaptive Substance Use

Another aspect of the invention is a method of assessing a subject's risk for maladaptive substance use. The method entails measuring one of several CCK_{B} receptor-related parameters in a biological sample from the subject. Suitable parameters include the levels of: CCK_{B} receptor ligands (e.g., CCK-4 or CCK-5), CCK_{B} receptor ligand precursors (e.g., pro-CCK or CCK-8), endoproteases involved in producing CCK_{B} receptor ligands, CCK_{B} receptors, receptor polynucleotides (e.g., mRNA), and function. The considerations affecting sample preparation and assay are as described above, with the additional consideration that sample collection is preferably minimally invasive to the subject.

The risk for maladaptive substance use is directly correlated with each of these levels. To determine whether the subject has a normal, elevated, or reduced risk, the level measured for the selected CCK_{B} receptor parameter is compared to that of an appropriate matched (e.g., for age, sex, etc.) control population. The control population can be representative of the general population to allow a determination of risk of the individual subject as compared to, for example, the average risk in the general population.

If a subject is determined to have a high risk for maladaptive substance use, a CCK_{B} receptor inhibitor can be administered to the subject to reduce this risk. Preferably, the inhibitor dose is sufficient to reduce the levels CCK_{B} receptors, receptor polynucleotides, and/or function to within a normal range (i.e., the range observed in the control population).

Kits

The invention also provides kits useful in practicing the methods of the invention. In one embodiment, a kit of the invention includes a CCK_{B} receptor inhibitor in a suitable container. In a variation of this embodiment, the CCK_{B} receptor inhibitor is formulated in a pharmaceutically acceptable carrier. The kit preferably includes instructions for administering the CCK_{B} receptor inhibitor to a subject to mitigate a symptom of maladaptive substance use.

In another embodiment, the kit is a diagnostic kit for use in assessing a subject's risk for maladaptive substance use. The kit includes at least one component that specifically binds to CCK_{B} polypeptides or polynucleotides. This binding component can be used to detect the presence of its binding partner in a biological sample from the subject. In a preferred embodiment, the binding component is labeled with a detectable label or, alternatively, the kit includes a labeling component that is capable of binding to, and thereby labeling, the binding component when the diagnostic method of the invention is carried out. The kit preferably includes instructions for carrying out the diagnostic method of the invention.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered to illustrate, but not to limit, the claimed invention.

### Example 1

CCK Antagonist Attenuates the Expression of Morphine Conditioned Place

Preference via Dopamine Receptors in the Anterior Nucleus Accumbens

Abstract

The present study was conducted to determine whether the CCK_{B} antagonist L-365, 260 could block the rewarding effects of morphine using the conditioned place preference paradigm and to determine whether the dopaminergic reward pathway contributes to this effect. This study demonstrates that systemic administration of the CCK_{B} antagonist L-365, 260 attenuates the expression of morphine conditioned place preference (CPP) and shows that this effect is mediated by CCK_{B} receptors in the anterior nucleus accumbens (NAcc). Additionally, this effect was found to be dependent on D₂ receptor activation in the anterior nucleus accumbens (NAcc). These results indicate that endogenous CCK modulates the rewarding effects of morphine associated cues and suggest that CCK antagonists may be useful in the treatment of drug craving and addiction.

Introduction

The conditioned place preference paradigm offers a substantive means of assessing the mechanism by which drug-associated cues control appetitive behaviors. In the current study, CPP was used to determine the contribution of CCK acting in the NAcc on expression of preference for an environment previously paired with morphine. The hypothesis that CCK contributes to the expression of the rewarding effects of morphine was examined. If so, administration of a CCK-B antagonist would decrease morphine CPP. Since the NAcc had been implicated in the expression of morphine CPP (Tzschentke, 1998), the role of dopamine in CCK actions at this site was investigated. Additionally, as previous groups have noted a difference in the actions of CCK in the anterior versus posterior NAcc (Vaccarino & Vaccarino, 1989; Marshall et al., 1991; Ladurelle et al, 1993), the possibility of differences in the effects of CCK drugs on expression of morphine CPP when injected into either the anterior or posterior region of the NAcc was examined. Lastly, because a change in affinity at the D₂ binding site has been noted in response to striatal CCK (Murphy & Schuster, 1982; Agnati et al., 1983), the possible role of dopamine D₂ receptors in the intra-accumbens effects of CCK on expression of morphine CPP was studied.

Methods

Subjects

68 male Sprague-Dawley rats (Charles River, Wilmington, Mass.) weighing 275-300 grams at the onset of the study were individually housed in a temperature controlled environment (70° F.) and kept on a 12 hour light/dark cycle. Rat chow and water were available ad libitum. Animals were tested at the same time during their light cycle each day. All experimental protocols were approved by the Institutional Animal Care and Use Committee and were conducted in accordance with the Guide for the Care and Use of Laboratory Animals (N1H). 22 rats were used for systemic injections and the remaining rats were cannulated for NAcc microinjections.

Surgery

Animals were anesthetized with a ketamine-xylazine mixture and maintained on isoflurane (0.5 liter/min) as needed for the duration of surgery. Animals were placed in a stereotaxic frame and were implanted with bilateral 26-gauge stainless steel chronic guide cannulae (Plastics One, Roanoke, V A) into either the Anterior (AP, 2.2; ML, ±1.1; DV, −5.5) or Posterior (AP, 1.2; ML, ±1.1; DV, −5.5) NAcc. Cannulae were secured to the skull with dental cement. At the end of the surgical procedure, animals were treated with penicillin and topical antibiotics. A stainless steel dummy cannula (Plastics One) was inserted into each guide cannula and remained in place when the guide cannulae were not in use. Animals were allowed a 1-week recovery period prior to behavioral testing.

Conditioned Place Preference

Animals were trained in 3 chamber place conditioning boxes (Med Associates) in which 2 chambers (28×21×21 cm) that differed in color (one black, one white), light level, and texture were separated by a neutral gray chamber (12×21×21). During the initial baseline period, animals were placed in the central chamber and were allowed to freely explore all 3 chambers for a period of 30 minutes. Beam breaks, entries, and time spent in each chamber were automatically recorded using infrared beams. Animals were excluded from the study if baseline data revealed a chamber bias of >250 seconds. During each conditioning session, animals were injected with either morphine or saline and were then immediately confined to one of the two larger chambers for 60 minutes. Rats received 2 conditioning sessions per day for a period of 4 days. Conditioning sessions were separated by a minimum period of 5 hours. Animals were tested for expression of conditioned place preference 1 day after the final conditioning session. Drug side and order were counter-balanced. This place preference procedure has been shown to induce a strong and specific place preference to morphine (Kim et al., 2004). Systemic L-365, 260 was administered 30 minutes before the onset of CPP testing.

Microinjections

All injection sites were based on the atlas of Paxinos and Watson (1998). Bilateral injections were made into either the Anterior (AP, 2.2; ML, +1.1; DV, −5.5) or Posterior (AP, 1.2; ML, ±1.1; DV, −5.5) NAcc. Each injection was made using a 1 μl syringe (Hamilton, Reno, Nev.) attached to 20 cm of PE 50 tubing connected to a 33-gauge injection cannula (Plastics One). Microinjections were given at a rate of 0.5 μl per minute using a syringe pump (kd Scientific, Holliston, M A). A volume of 0.5 μl was injected into each side of the NAcc. Injection cannulae extended 2 mm beyond guide cannulae and were left in place for 1 minute following microinjections to minimize the backflow of drug solution. 10 minutes after microinjection was completed, animals were placed into the central CPP chamber and allowed to explore for a period of 30 minutes. Animals received a maximum of four bilateral NAcc microinjections. The number of microinjections each animal received was determined by the persistence of place preference. Participation was terminated if an animal's preference score did not return to the pre-injection level within 24 hours following a microinjection. Microinjection drug order was counterbalanced and began a minimum of one day following the test for expression of morphine CPP. Animals were tested for baseline expression of morphine CPP 24 hours following each microinjection to ensure the return to pre-injection baseline. At the conclusion of the experiment, animals were anesthetized with pentobarbital and perfused intracardially through the ascending aorta with 0.1M phosphate buffered saline followed by 10% formalin. Brains were sectioned coronally at 50 μm, mounted and stained with neutral red.

Drugs

Morphine sulfate powder was the gift of the NIDA Drug Supply Program (Research Triangle Park, N C) and was dissolved in physiological saline and injected at a dose of 10 mg/kg s.c. L-365, 260 was the gift of ML Laboratories (St. Albans, UK) and was dissolved in 5% DMSO. L-365, 260 was injected i.p. at a dose of 1 mg/kg and was microinjected at a dose of 15 ng/site. CCK-4 (100 ng/site), CCK-8 (100 ng/site), lorglumide (500 ng/site), and raclopride (4 μg/site) were all obtained from Research Biochemicals (Natick, M A) and were dissolved in physiological saline.

Statistical Analysis

*Difference scores were calculated by subtracting the time spent in the saline paired chamber from the time spent in the morphine paired chamber during a test session. A positive score is therefore indicative of place preference while a negative score would indicate place aversion. Values are given as arithmetic means ±SEM. Statistical significance was set at p<0.05. Paired comparisons between groups were conducted using a Student's t-test (two-tailed). All statistical tests were parametric and were conducted using commercially available software (Excel).

Results

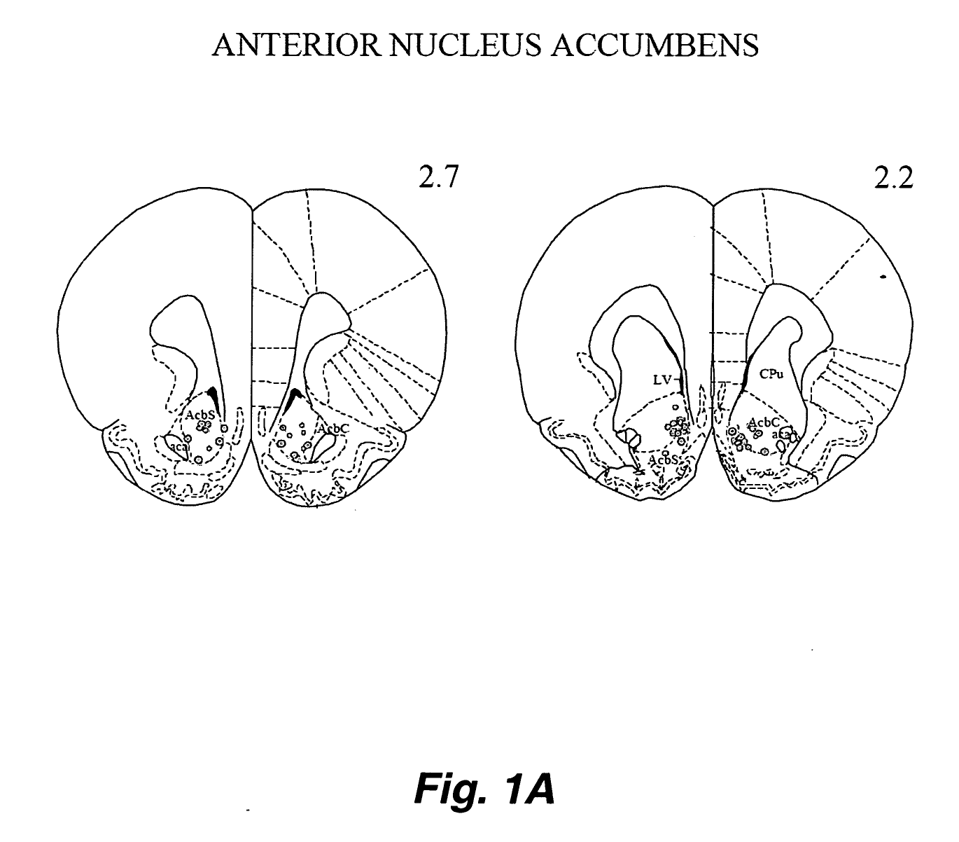

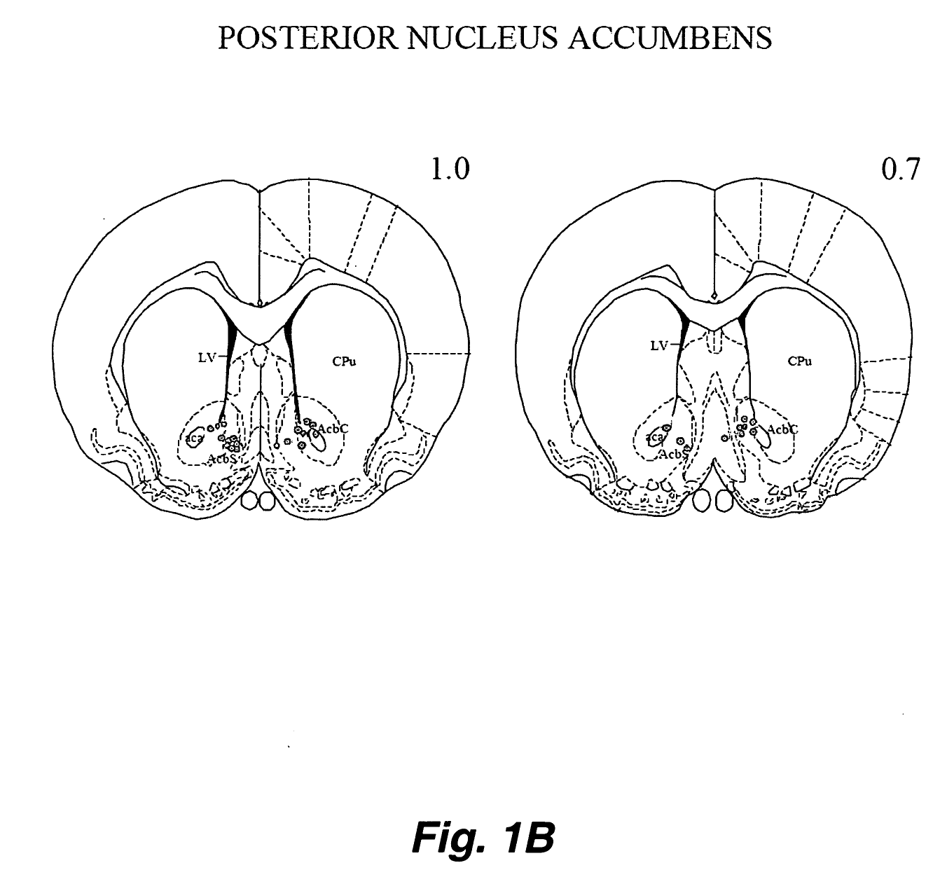

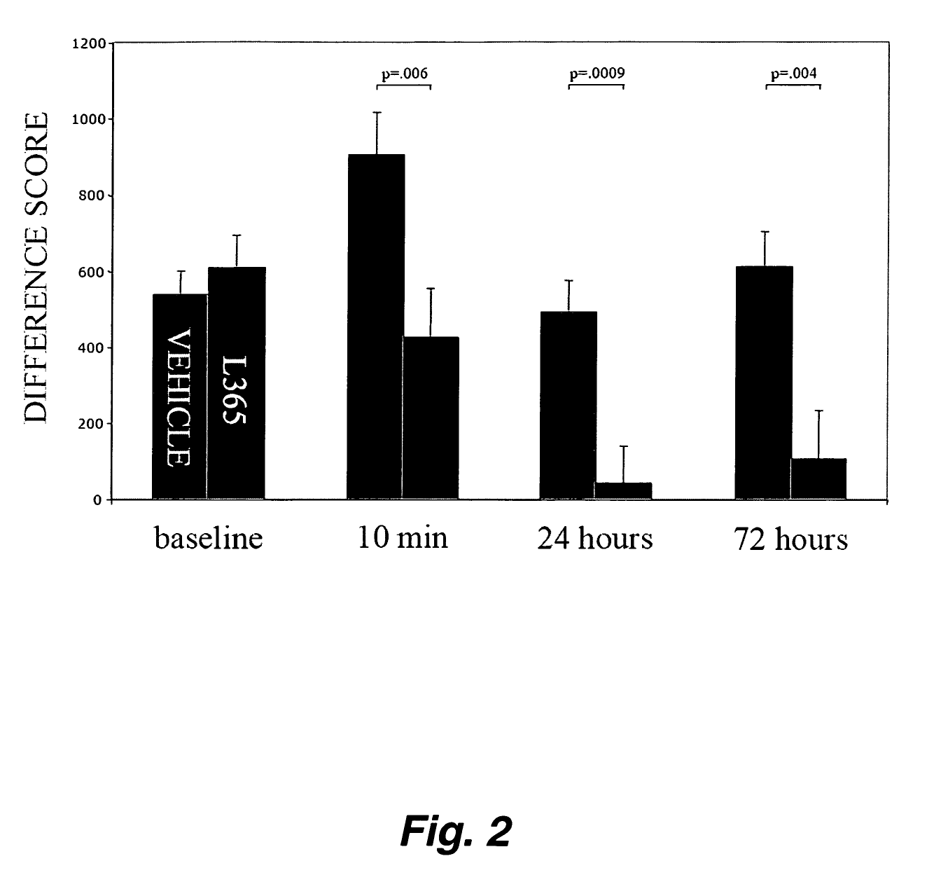

NAcc microinjection sites are illustrated in FIG. 1. When administered systemically following the acquisition of conditioned place preference, the CCK_{B} specific antagonist L-365, 260 (1 mg/kg) attenuated the expression of morphine CPP (FIG. 2). Interestingly, this effect was not only significant when animals were tested immediately after injection (p=0.006), but also when animals were tested 24 (p=0.0009) and 72 (p=0.004) hours after antagonist injection. This indicates a prolonged change in the expression of morphine conditioned place preference. These data suggest that CCK is important for the expression of the rewarding effects of morphine in the conditioned place preference paradigm.

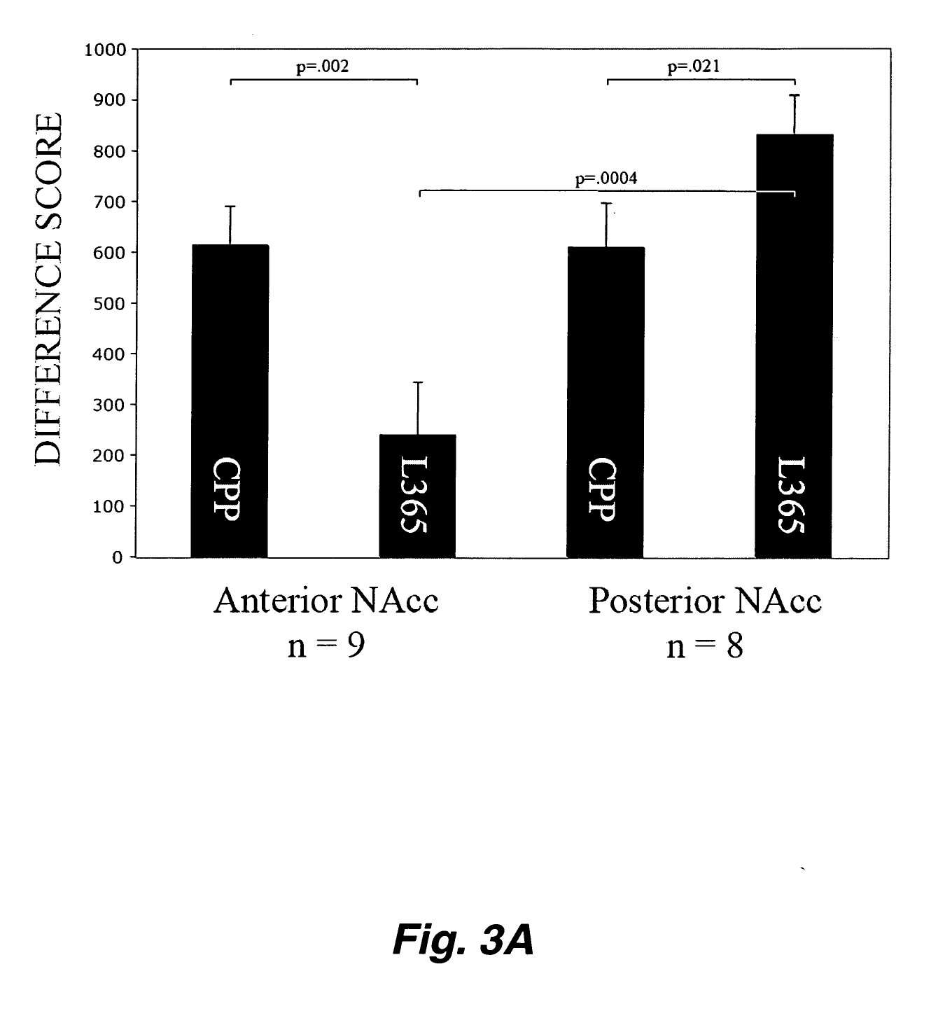

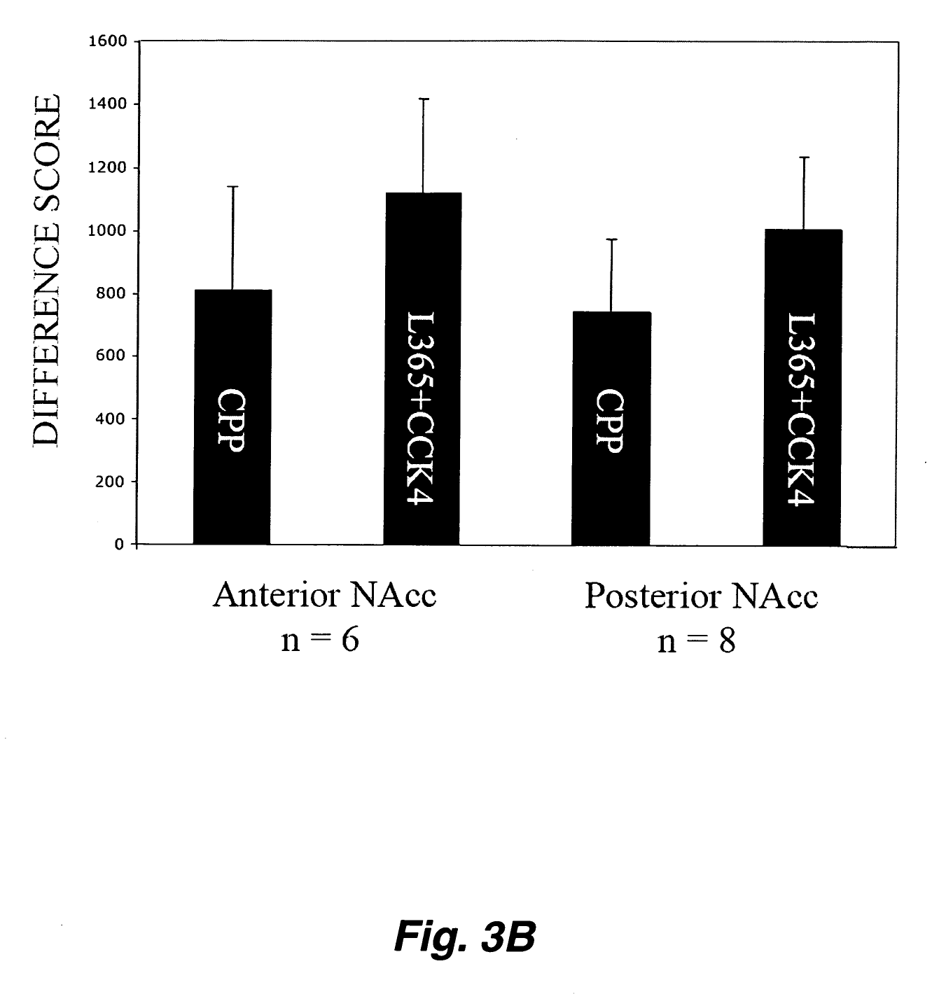

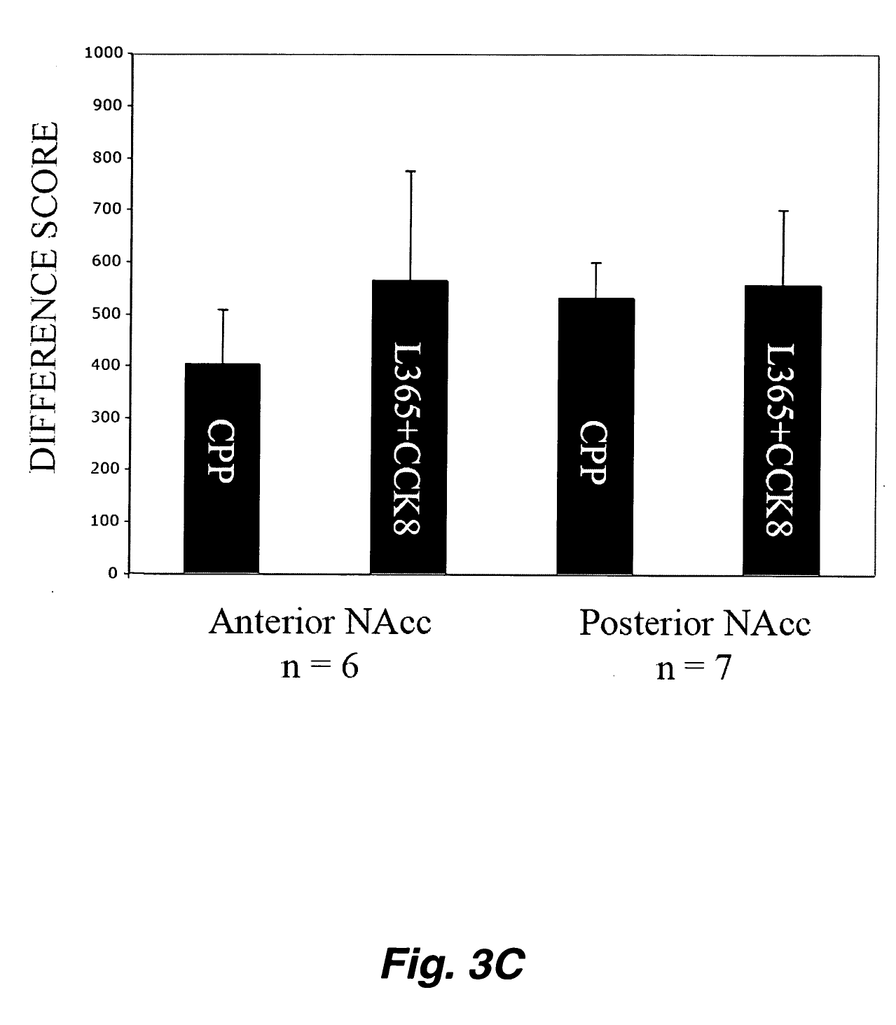

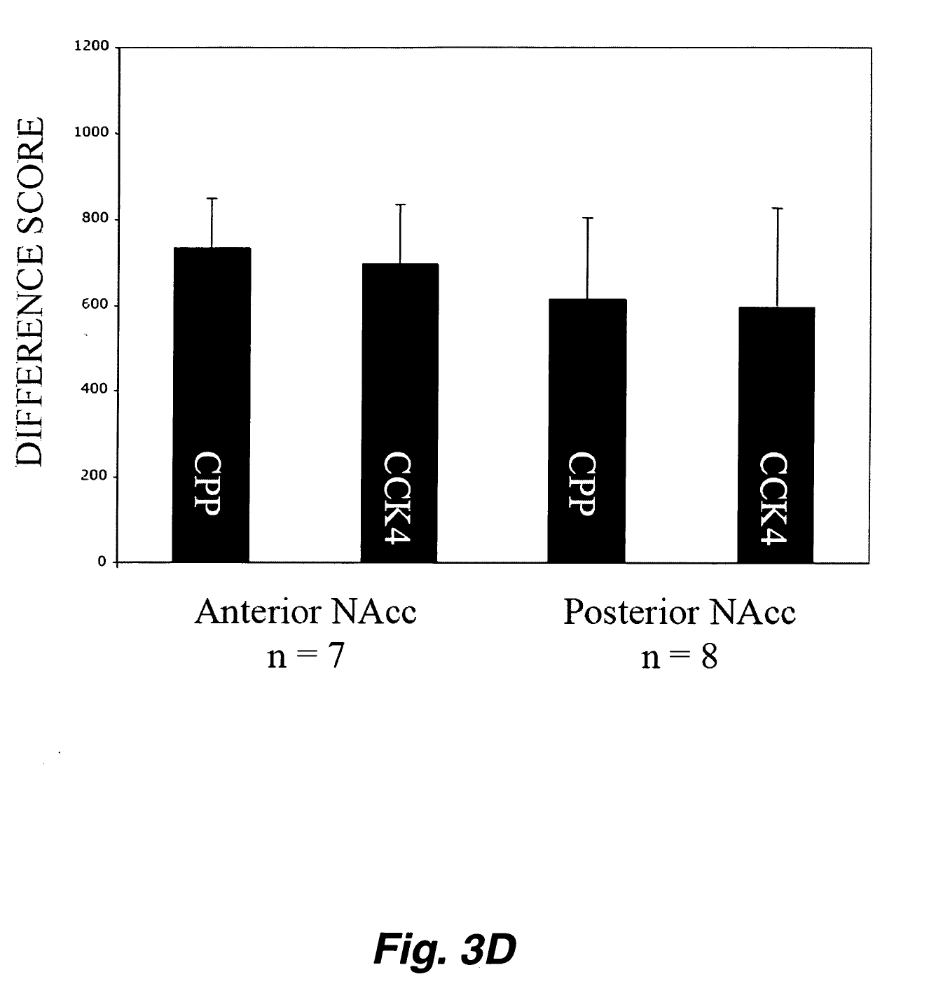

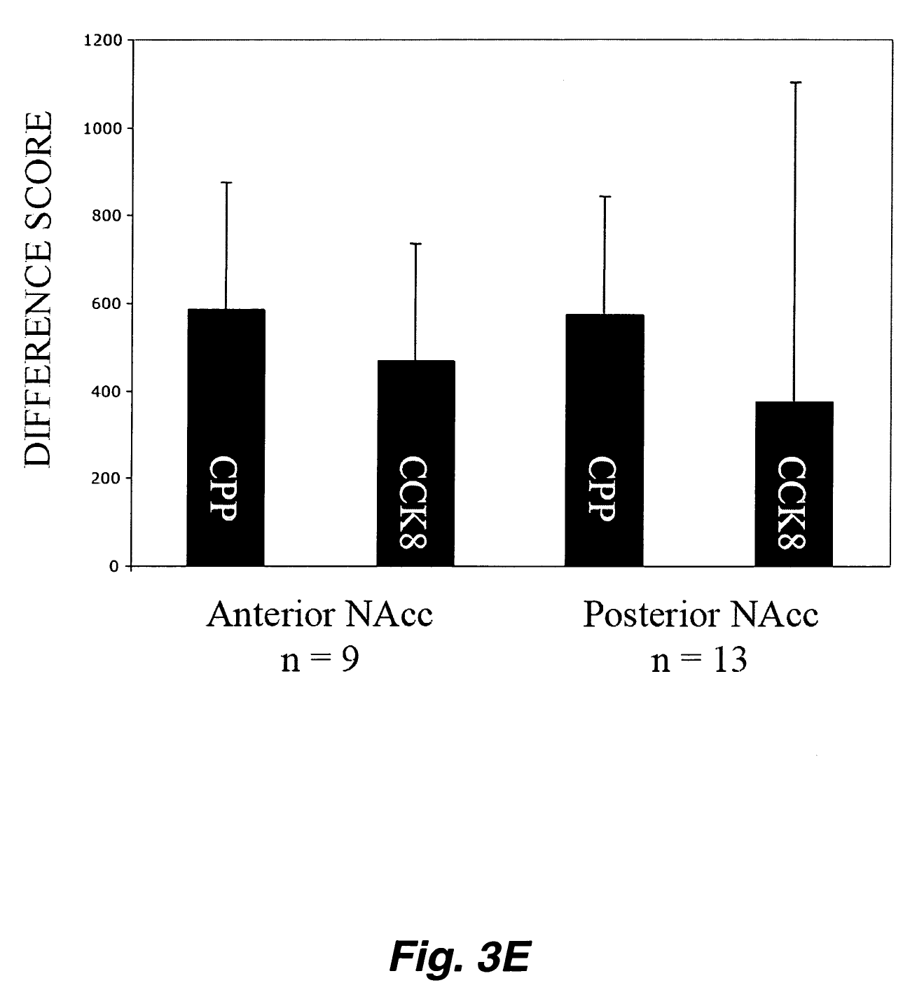

In order to determine the contribution of CNS reward circuitry to this effect, L-365, 260 (1 ng) was microinjected into either the anterior or posterior NAcc (FIG. 3A). L-365, 260 microinjected into the anterior NAcc attenuated the expression of morphine CPP (p=0.002) while L-365, 260 microinjected into the posterior NAcc potentiated the expression of morphine CPP (p=0.021). Both effects were reversed by the co-injection of either CCK-4 (10 ng; FIG. 3B) or CCK-8 (100 ng; FIG. 3C), neither of which had an effect on the expression of morphine CPP when injected independently (FIG. 3D, E).

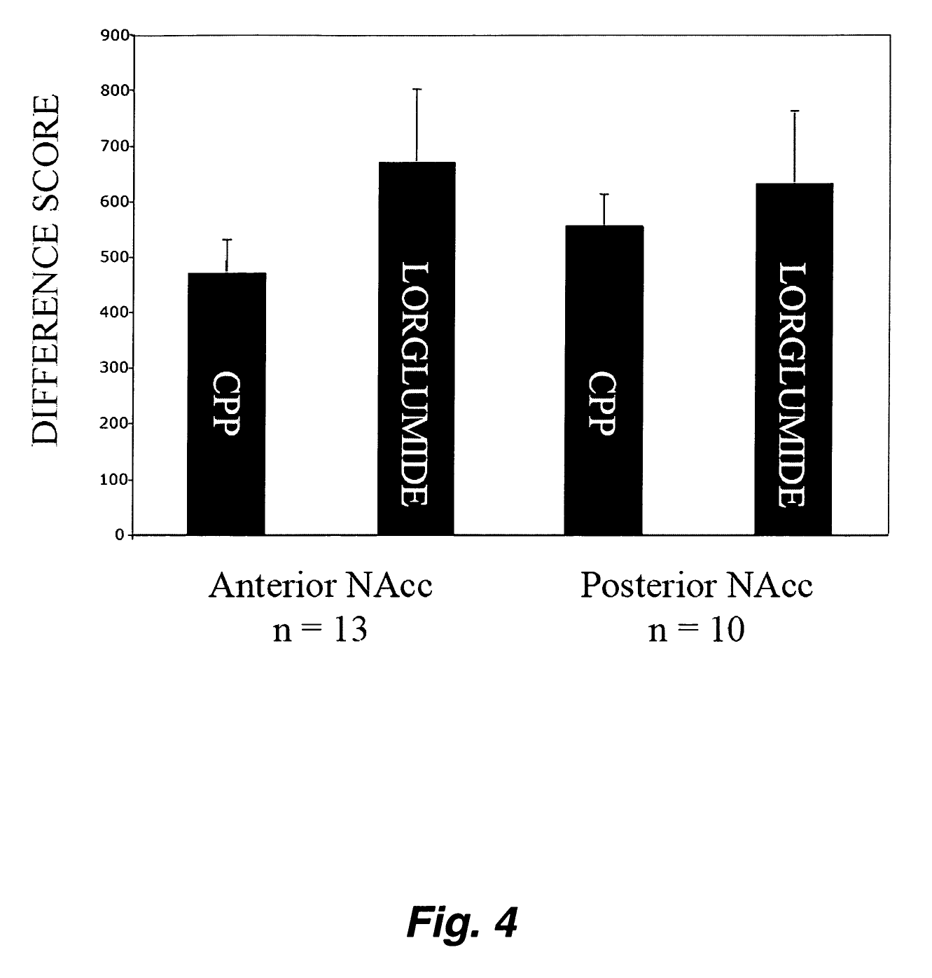

To determine the specificity of the L-365, 260 effect within the NAcc, the specific CCK_{A} antagonist lorglumide was microinjected into either the anterior or posterior NAcc (FIG. 4). Lorglumide (lSng) had no effect on morphine CPP in either the anterior or posterior NAcc, which corroborates the results of previous in situ hybridization studies in suggesting the absence of the CCK_{A} receptor in the NAcc, and indicates that the effect of CCK on expression of morphine CPP in the NAcc is mediated primarily through the CCK_{B} receptor.

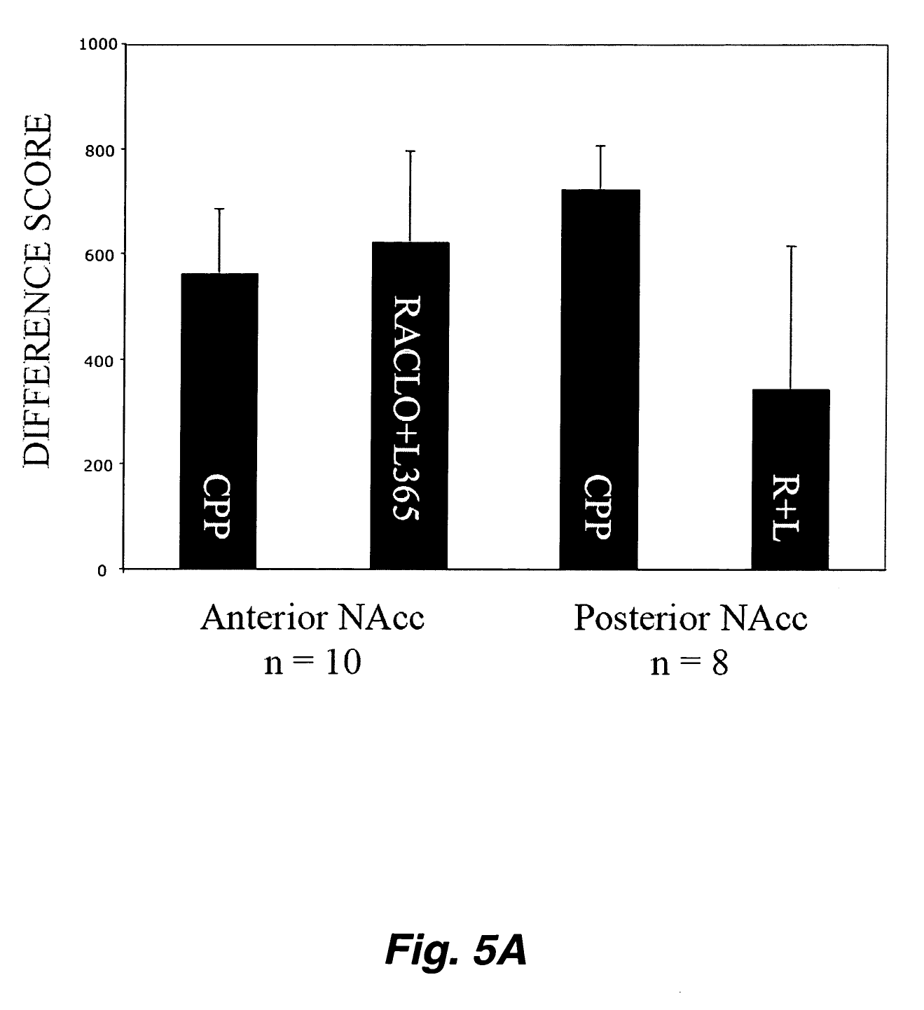

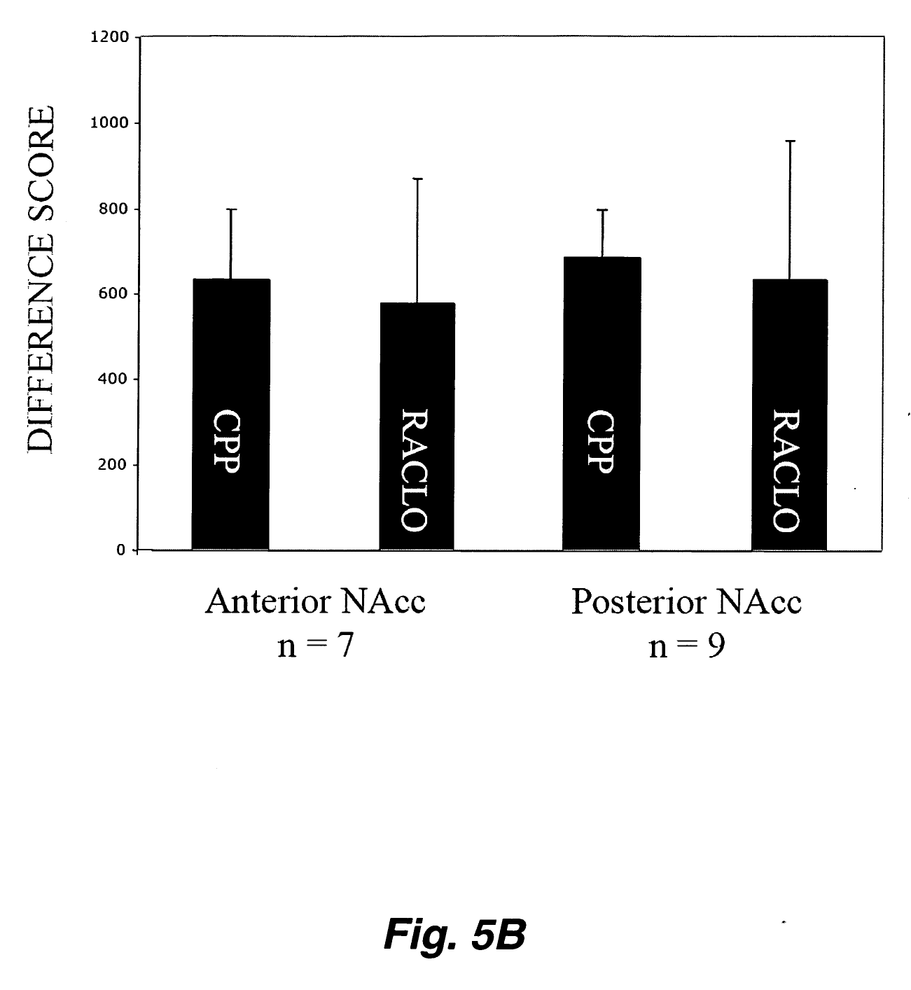

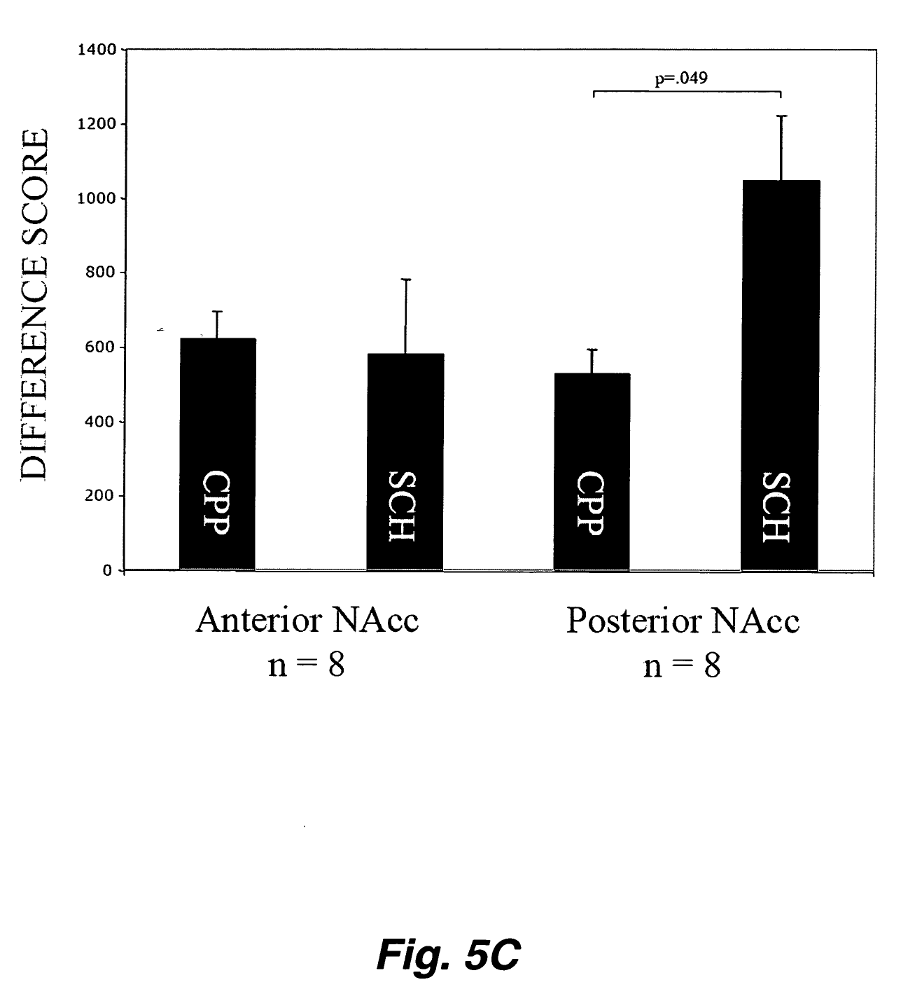

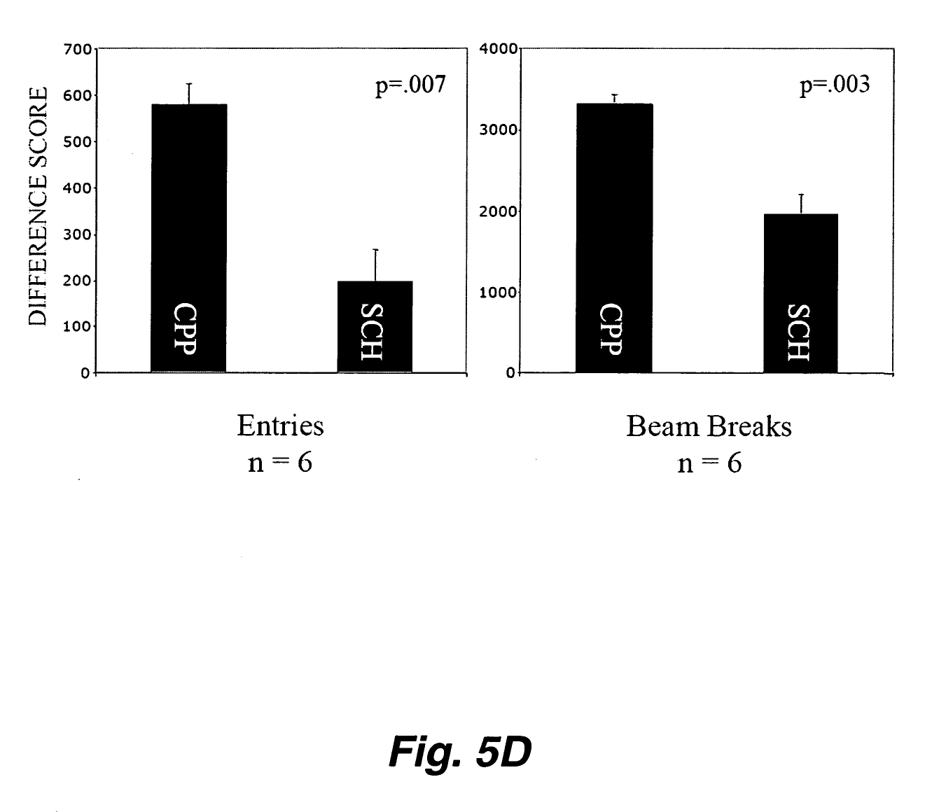

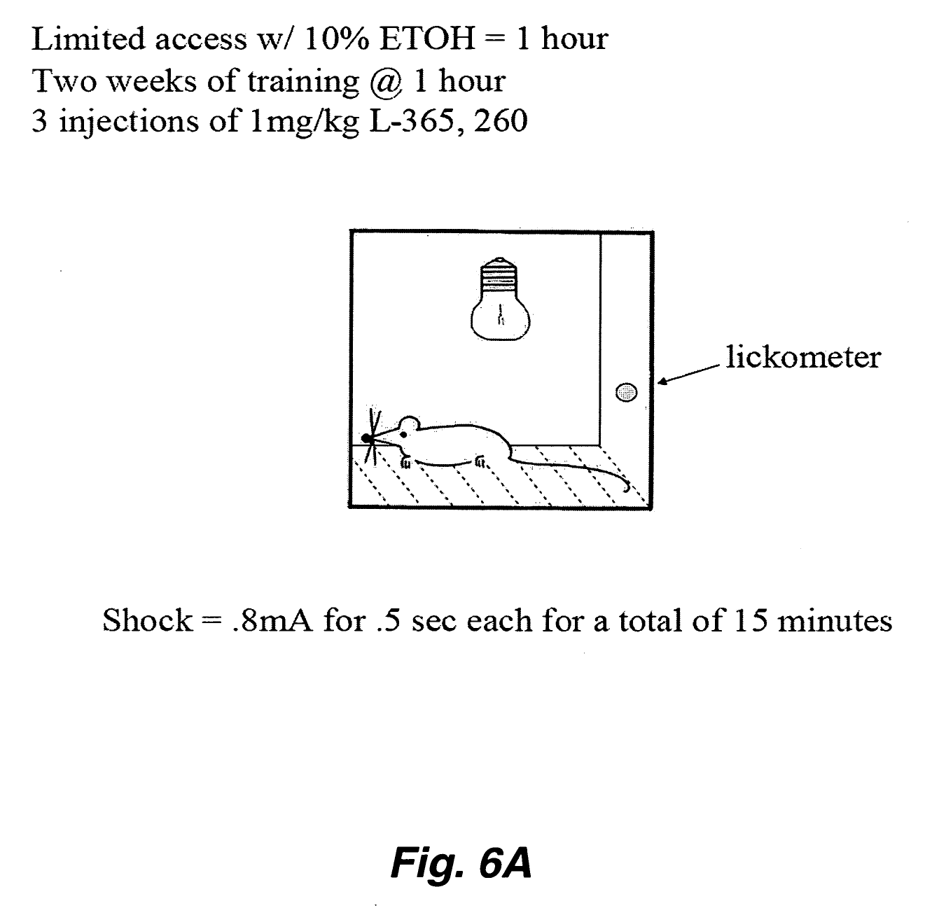

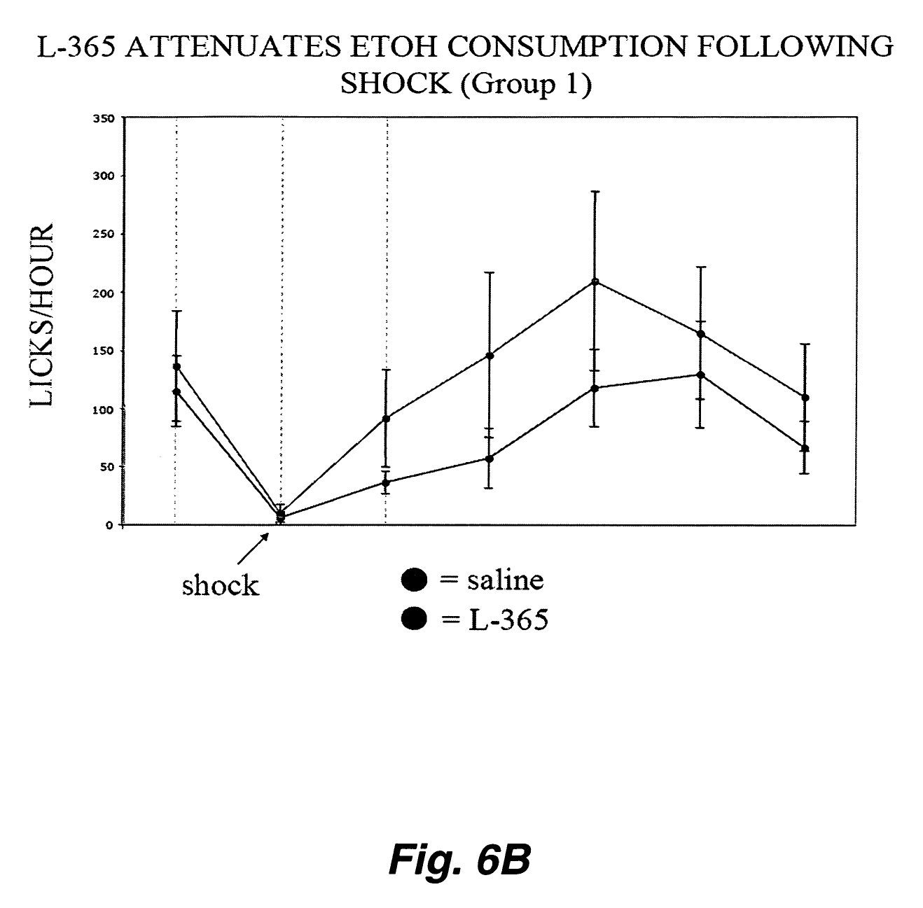

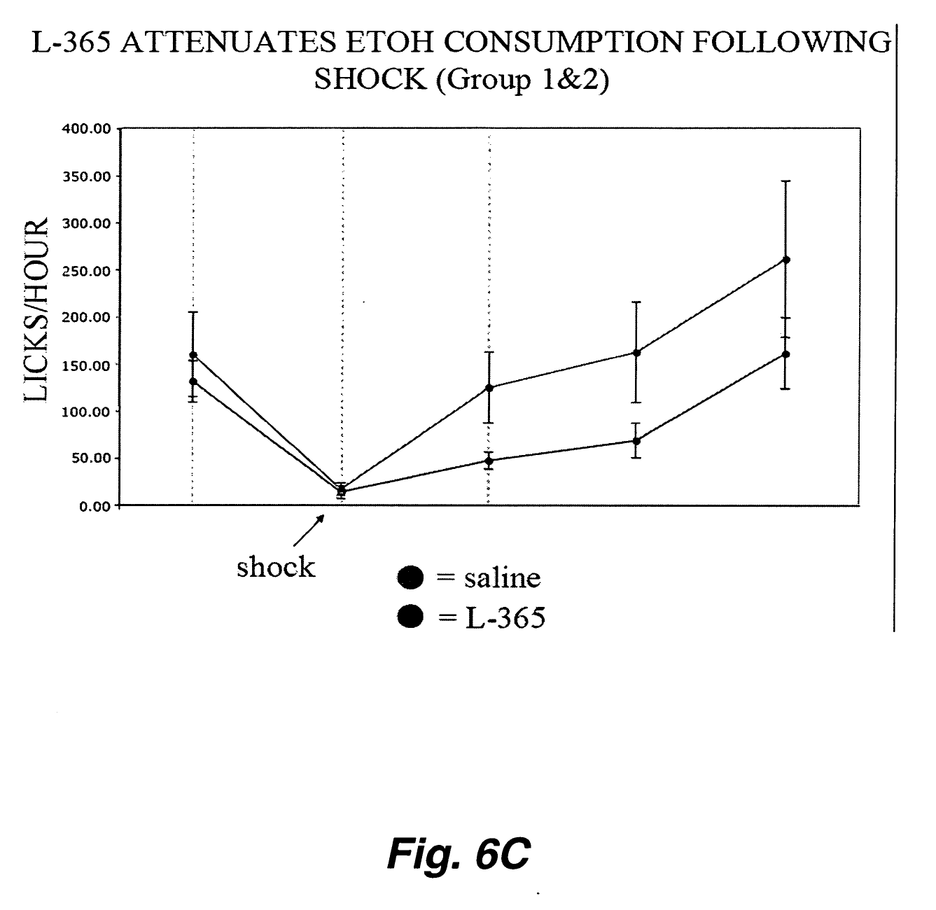

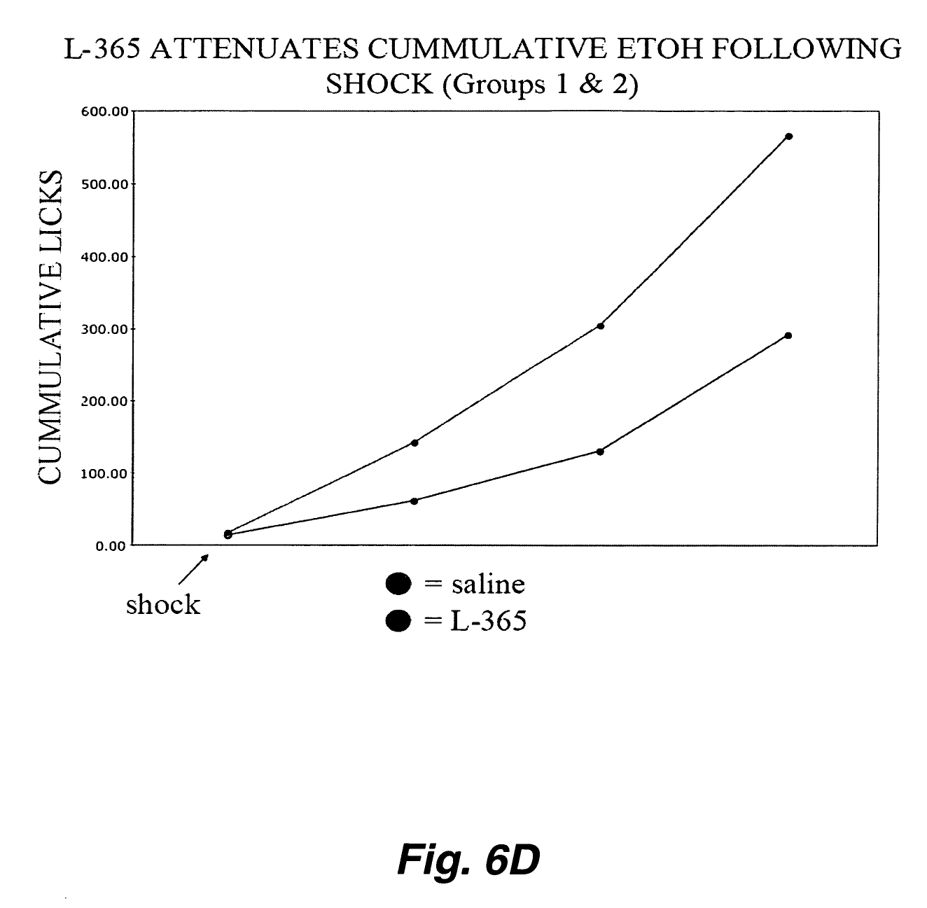

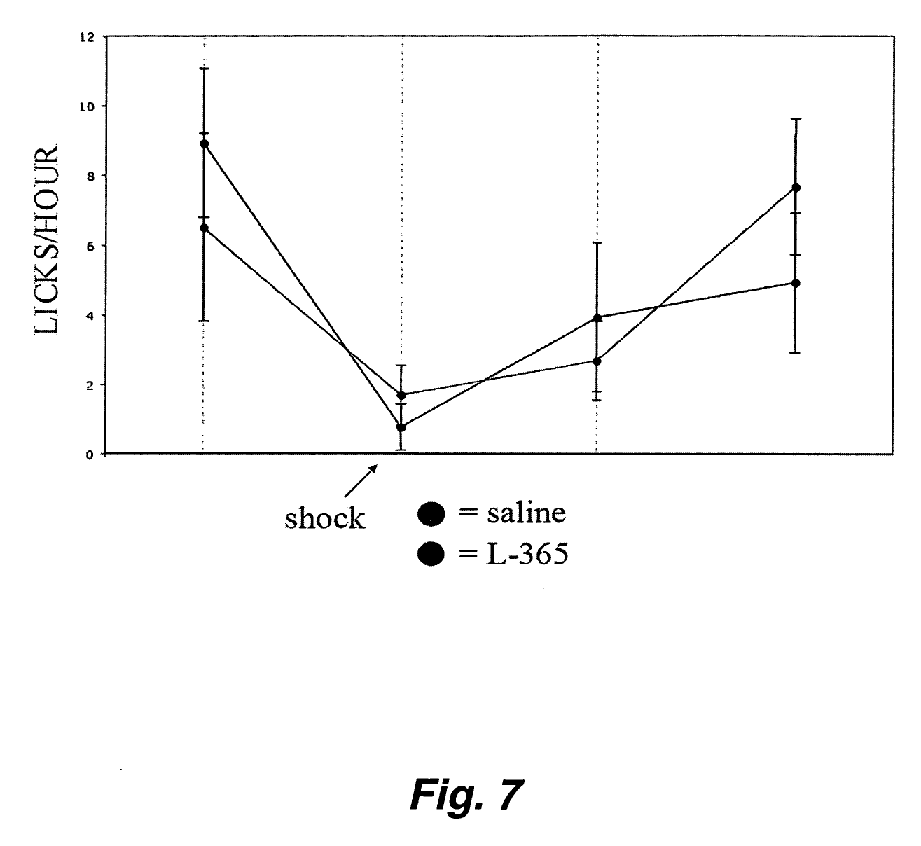

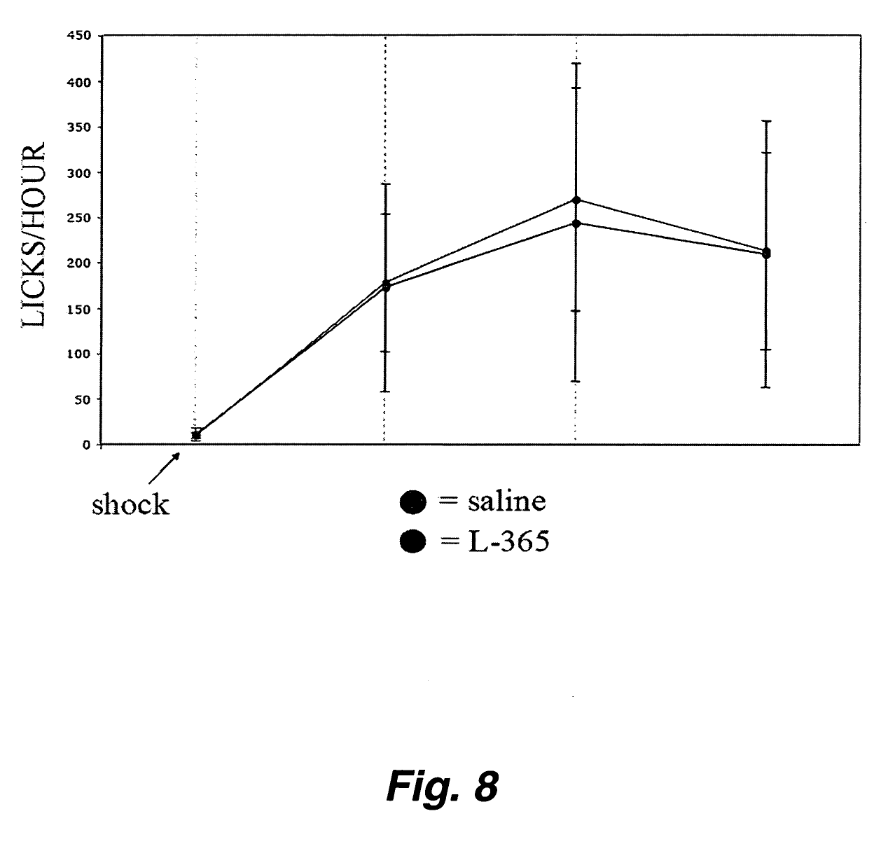

In order to ascertain the contribution of the dopamine system to the effects of CCK on expression of morphine CPP, the D₂ specific antagonist raclopride was microinjected into the anterior and posterior NAcc. Raclopride (4 μg) had no effect on morphine CPP when microinjected independently into either the anterior or posterior NAcc (FIG. 5B) but did reverse the effects of L-365, 260 in both regions (FIG. 5A) suggesting that the effect of CCK on expression of morphine CPP is mediated by dopamine acting at the D₂ receptor. In contrast, the D₁ specific antagonist SCH-23390 (2 μg) potentiated morphine CPP when microinjected independently into the posterior, but not the anterior, NAcc (5C). Graphing the average number of room entries and beam breaks following posterior microinjections revealed a significant decrease in locomotion following SCH-23390 administration (SD). Therefore, it is possible that the effects of SCH-23390 on expression of morphine CPP are secondary to effects on motor behavior. Because of this confound, SCH-23390 was not co-injected with L-365, 260.

Discussion

The current study shows that systemic injection of the CCK_{B} antagonist L-365, 260 attenuates the expression of morphine conditioned place preference and that this effect involves an action at the D₂ receptor in the anterior, but not the posterior, nucleus accumbens. Previous work demonstrated that activation of D₂ presynaptic autoreceptors decreases nucleus accumbens dopamine release (Dugast et al., 1997; Phillips et al., 2002). Additionally, CCK acts at CCK_{B} receptors in the nucleus accumbens to decrease D₂ receptor binding (Murphy & Schuster, 1982; Agnati et al. 1983; Li et al. 1995). Consequently, reduction of this D₂ autoreceptor-mediated inhibition by a CCK_{B} agonist might be expected to increase dopamine release (Ladurelle et al., 1993; Ferraro et al. 1996). Consistent with this idea, CCK_{B} and D₂ receptor mRNAs are co-localized within neurons in the caudate putamen (Hansson et al., 1998). The current study indicates that a similar mechanism may be regulating the expression of morphine conditioned place preference (CPP) in the anterior NAcc. By increasing binding affinity at D₂ binding sites, a CCK_{B} antagonist would decrease the amount of dopamine released from presynaptic terminals. This decrease in dopamine release translates into a decreased reward value associated with sensory cues in the morphine-paired chamber, resulting in an attenuation in the expression of morphine CPP.

Microdialysis studies suggest that under basal conditions there is tonic release of CCK in the NAcc (Maidment et al., 1991; You et al., 1994), which might explain the lack of a consistent CCK agonist effect. If CCK receptors are already saturated, microinjection of additional CCK should be unable to alter expression of CPP. Under the present conditions, the CCK antagonist seems to be acting as a brake on expression of morphine CPP suggesting an action on dopamine dependent incentive cues (Yun et al., 2004). Perhaps by decreasing dopamine release, a CCK_{B} antagonist decreases the value of incentive cues in a drug-paired environment.

The anterior NAcc receives a projection from the prefrontal cortex that includes CCK containing glutamatergic neurons (Meyer et al., 1982; Morino et al., 1994). In contrast, the medial-posterior NAcc receives CCK projections that originate from the VTA and co-contain dopamine (Hokfelt et. al, 1980). Interestingly, the VTA dopaminergic projection to the anterior NAcc does not appear to contain CCK (Hokfelt et al., 1980; Seroogy et al., 1989; Ladurelle et al., 1993). Furthermore, the CCK containing projection from the VTA terminates in the NAcc shell while the CCK containing dopamine projection from the substantia nigra pars compacta terminates in the NAcc core (Lanca et al., 1998). Together with the present results, these findings are consistent with the idea that in the anterior NAcc shell CCK released from the terminals of prefrontal cortex neurons acts presynaptically at CCK_{B} receptors on the terminals of dopaminergic fibers that originate in the VTA and enhances dopamine release.

The neural circuits mediating drug reward include the VTA and NAcc. In addition to these structures, drug craving and relapse have been proposed to involve the prefrontal cortex, amygdala, and HPA axis (Erb et al., 1998; McLaughlin & See, 2003). The results of the current study indicate that the decreased expression of morphine CPP following L-365, 260 administration may be due to a change in the reinforcing or reward predictive properties of the drug associated context. This change appears to involve a mechanism within the anterior nucleus accumbens, which receives CCK projections from brain regions involved in craving and relapse.

In contrast with the present results, several groups have noted effects on locomotion, exploration, and learning of conditioned reward following the infusion of either CCK-8 or CCK_{A} antagonists into the posterior NAcc (Crawley, 1992; Derrien et al., 1993; Ladurelle et al., 1993; Josselyn et al., 1996). Additionally, CCK antagonizes the synaptic effects of dopamine in posterior NAcc slices in vitro (Yim & Mogenson, 1991). These results are also difficult to reconcile with the evidence that CCK_{A} receptors are absent in the posterior NAcc (Honda et al. 1993; Zajac et al., 1996; Lodge & Lawrence, 2001). One possible explanation is that microinjected CCK is able to diffuse into the anterior accumbens where it then acts on CCK_{B} receptors. Other explanations include the possibility that there are additional novel CCK receptors, or that autoradiography is not the most effective method for visualizing the distribution of CCK_{A} receptors within the NAcc and that immunohistochemical data might be more sensitive (Mercer & Beart, 2004).

A difference in the effects of CCK compounds in the anterior versus posterior NAcc has been noted previously (Vaccarino & Vaccarino, 1989; Marshall et al., 1991; Ladurelle et al, 1993). One possible explanation for this dichotomy is a difference in CCK receptor localization between the anterior and posterior NAcc. Previous studies have established that the CCK_{B} receptor is localized both pre and postsynaptically (Ferraro et al., 1996), yet no information currently exists on the localization of CCK_{B} receptors within subregions of the NAcc. The results of the current study could be explained by a presynaptic CCK_{B} receptor effect on dopaminergic neurons in the anterior NAcc and a postsynaptic CCK_{B} receptor effect on GABAergic neurons in the medial-posterior NAcc. Indeed, Tanganelli et al. (2001) have suggested a similar model to explain the modulatory effects of CCK on pre and post synaptic D₂ receptors in the NAcc.

L-365, 260 cannot be differentiated from saline in animal discrimination studies (Jackson et al., 1994) and has been safely administered to humans with no significant side effects (Murphy et al., 1993; Sramek, 1994; Bradwejn et al., 1994; Kramer et al., 1995; Lines et al. 1995; Grasing et al., 1996; van Megen et al., 1996; Bertoni et al., 2002; McCleane, 2002; McCleane, 2003), thus making it a good compound for human drug abuse treatment.

In conclusion, the CCK_{B} antagonist L-365, 260 significantly attenuates the expression of morphine conditioned place preference through a mechanism involving D₂ receptors in the anterior nucleus accumbens, and this attenuation can be long lasting. This result indicates that an active modulatory effect of endogenous CCK enhances the incentive value of morphine-associated cues. Consequently, CCK_{B} receptor antagonists are expected to be useful therapeutics in the treatment of drug abuse and cue induced craving.

### Example 2

### CCK Antagonist Attenuates Ethanol Consumption Following Shock

Rats (n=18) were administered 3 daily doses of systemic L-365 (1 mg/kg) prior to a one-hour ethanol (10%) access session. On the second day of L-365, 260 administration, animals were administered 0.8 mA of shock (0.5 seconds every 40 seconds for a period of 15 minutes) in ethanol access chambers directly prior to the onset of their one-hour access session. Animals receiving L-365, 260 consumed significantly less ethanol than saline controls on the two days following shock. These data can also be seen as cumulative curves charting total ethanol consumption for ethanol and saline control animals. This was a randomized cross over design with each animal serving as its own control.

L-365, 260 had no effect on ethanol consumption when shock was administered during extinction (n=12). Animals were administered 3 daily doses of systemic L-365, 260 after ethanol consumption was extinguished. On the second day of L-365, 260 administration, animals were administered 0.8 mA of shock (0.5 seconds every 40 seconds for a period of 15 minutes) in the ethanol access chambers directly prior to the onset of their one-hour extinction session. This was a randomized cross over design with each animal serving as its own control.

L-365 had no effect on ethanol reinstatement when shock was administered on the first day of re-initiation of ethanol access (n=12). Animals were administered 3 daily doses of systemic L-365, 260 after ethanol consumption was extinguished. On the first day of L-365, 260 administration, animals were administered 0.8 mA of shock (0.5 seconds every 40 seconds for a period of 15 minutes) in the ethanol access chambers directly prior to the onset of their one-hour reinstatement session. This was a randomized cross over design with each animal serving as its own control.

### REFERENCES

Agnati L F, Celani M F, Fuxe K. (1983) Cholecystokinin peptides in vitro modulate the characteristics of the striatal 3H-N-propylnorapomorphine sites. Acta Physiol Scand. 118(1): 79-81.
Baber N S, Dourish C T, Hill D R (1989) The role of CCK caerulein, and CCK antagonists in nociception. Pain. (3): 307-328.
Bertoni M, Olivieri F, manghetti M, Boccolini E, Bellomini M G, Blandizzi C, Bonino F, Del Tacca M. (2002) Effects of a bicarbonate-alkaline mineral water on gastric functions and functional dyspepsia: a preclinical and clinical study. Pharmacological Research. 46(6): 525-531.
Bhatnagar S, Viau V, Chu A, Soriano L, Meijer O C, Dallman M F. (2000) A cholecystokinin-mediated pathway to the paraventricular thalamus is recruited in chronically stressed rats and regulates hypothalamic-pituitary-adrenal function. Journal of Neuroscience. 20(14): 5564-5573.
Bradwejn J, Koszycki D, Couetoux du Tertre A, van Megen H, den Boer J, Westenberg H. (1994) The panicogenic effects of cholecystokinin-tetrapeptide are antagonized by L-365,260, a central cholecystokinin receptor antagonist, in patients with panic disorder. Arch Gen Psychiatry. 51(6): 486-493.
Brog J S, Salyapongse A, Deutch A Y, Zahm D S. (1993) The patterns of afferent innervation of the core and shell in the “accumbens” part of the rat ventral striatum: immunohistochemical detection of retrogradely transported fluoro-gold. J Comp Neurol. 338(2): 255-278.
Chang R S, Chen T B, Bock M G, Freidinger R M, Chen R, Rosegay A, Lotti V J (1989) Characterization of the binding of [3H]L-365,260: a new potent and selective brain cholecystokinin (CCK-B) and gastrin receptor antagonist radioligand. Mol Pharmacol. 35(6): 803-8.
Crawley J N. (1992) Subtype-selective cholecystokinin receptor antagonists block cholecystokinin modulation of dopamine-mediated behaviors in the rat mesolimbic pathway. J. Neurosci. 12(9): 3380-3391.
Crawley J N, Corwin R L. (1994) Biological actions of cholecystokinin. Peptides. 15(4): 731-755.
Crespi F. (1998) The role of cholecystokinin (CCK), CCK-A or CCK-B receptor antagonists in the spontaneous preference for drugs of abuse (alcohol or cocaine) in naive rats. Methods Find Exp Clin Pharmacol. (8): 679-697.
Derrien M, Durieux C, Dauge V, Roques B P. (1993) Involvement of D2 dopaminergic receptors in the emotional and motivational responses induced by injection of CCK-8 in the posterior part of the rat nucleus accumbens. Brain Res. 617(2): 181-188.
Di Ciano P. Everitt B J. (2004) Direct interactions between the basolateral amygdala and nucleus accumbens core underlie cocaine-seeking behavior by rats. J Neurosci. 24(32): 7167-7173.
Dourish C T, Hawley D, Iversen S D. (1988) Enhancement of morphine analgesia and prevention of morphine tolerance in the rat by the cholecystokinin antagonist L-364,718. Eur J Pharmacol. 147(3): 469-472.
Dugast C, Brun P, Sotty F, Renaud B, Suaud-Chagny M F. (1997) On the involvement of a tonic dopamine D2-autoinhibition in the regulation of pulse-to-pulse-evoked dopamine release in the rat striatum in vivo. Naunyn Schmiedebergs Arch Pharmacol. 355(6): 716-719.
Erb S, Shaham Y, Stewart J. (1998) The role of corticotropin-releasing factor and corticosterone in stress- and cocaine-induced relapse to cocaine seeking in rats. J Neurosci. 18(14): 5529-5536.
Faris P L, Komisaruk B R, Watkins L R, Mayer D J. (1983) Evidence for the neuropeptide cholecystokinin as an antagonist of opiate analgesia. Science. 219(4582): 310-312.
Ferraro L, O'Connor W T, Li X M, Rimondini R, Beani L, Ungerstedt U, Fuxe K, Tanganelli S. (1996) Evidence for a differential cholecystokinin-B and -A receptor regulation of GABA release in the rat nucleus accumbens mediated via dopaminergic and cholinergic mechanisms. Neuroscience. 73(4): 941-950.
Grasing K, Freedholm D, Murphy M G, Swigar M, Russer T, Nosher J, Lin J, Frame V, Clarke L, Seibold J R. (1996) Selective blockade of cholecystokinin type B receptors with L-365,260 does not impair gallbladder contraction in normal humans. Am J Gastroenterol. 91(3): 569-573.
Hamilton M E, Redondo J L, Freeman A S. (2000) Overflow of dopamine and cholecystokinin in rat nucleus accumbens in response to acute drug administration. Synapse. 38(3): 238-242.
Hansson A C, Andersson A, Tinner B, Cui X, Sommer W, Fuxe K. (1998) Existence of striatal nerve cells coexpressing CCK(B) and D2 receptor mRNAs. Neuroreport. 9(9): 2035-2038.
Higgins G A, Nguyen P, Sellers E M (1992) Morphine place conditioning is differentially affected by CCK_{A} and CCK_{B} receptor antagonists. Brain Research. 572(1-2): 208-215.
Hokfelt T, Skirboll L, Rehfeld J F, Goldstein M, Markey K, Dann O. (1980) A subpopulation of mesencephalic dopamine neurons projecting to limbic areas contains a cholecystokinin-like peptide: evidence from immunohistochemistry combined with retrograde tracing. Neuroscience. 5(12): 2093-2124.
Hökfelt T, Herrera-Marschitz M, Seroogy K, Ju G, Staines W A, Holets V, Schalling M, Ungerstedt U, Post C, Rehfeld J F, Frey P, Fischer J, Dockray G J, Hamaoka T, Walsh J H, Goldstein M (1988) Immunohistochemical studies on cholecystokinin (CCK)-immunoreactive neurons in the rat using sequence specific antisera and with special reference to the caudate nucleus and primary sensory neurons. J Chem Neuroanat. 1:11-52.
Honda T, Wada E, Battey J F and Wank S A (1993) Differential gene expression of CCK_{A} and CCK_{B} receptors in the rat brain. Mol. Cell. Neurosci. 4: 143-154. Jackson A, Tattersall D, Bentley G, Rycroft W, Bourson A, Hargreaves R, Tricklebank M, Iversen S. (1994) An investigation into the discriminative stimulus and reinforcing properties of the CCK_{B}-receptor antagonist, L-365,260 in rats. Neuropeptides. 26(5): 343-353.
Josselyn S A, Franco V P, Vaccarino F J (1996) Devazepide, a CCK_{A} receptor antagonist, impairs the acquisition of conditioned reward and conditioned activity. Psychopharmacology. 123(2): 131-43.
Katsuura G, Itoh S. (1985) Potentiation of beta-endorphin effects by proglumide in rats. Eur J Pharmacol. 107(3): 363-366.
Kim J A, Pollak K A, Hjelmstad G O, Fields H L (2004) A single cocaine exposure enhances both opioid reward and aversion through a ventral tegmental area-dependent mechanism. Proc Natl Acad Sci 101(15): 5664-5669.
Kramer M S, Cutler N R, Ballenger J C, Patterson W M, Mendels J, Chenault A, Shrivastava R, Matzura-Wolfe D, Lines C, Reines S. (1995) A placebo-controlled trial of L-365,260, a CCK_{B} antagonist, in panic disorder. Biological Psychiatry. 37(7): 462-466.
Ladurelle N, Keller G, Roques B P, Dauge V. (1993) Effects of CCK8 and of the CCK_{B}-selective agonist BC264 on extracellular dopamine content in the anterior and posterior nucleus accumbens: a microdialysis study in freely moving rats. Brain Res. 628(1-2): 254-262.
Lanca A J, De Cabo C, Arifuzzaman A I, Vaccarino F J. (1998) Cholecystokinergic innervation of nucleus accumbens subregions. Peptides. 19(5): 859-868.
Lavigne G J, Millington W R, Mueller G P (1992) The CCK-A and CCK-B receptor antagonists, devazepide and L-365,260, enhance morphine antinociception only in non-acclimated rats exposed to a novel environment. Neuropeptides. 21(2): 119-29.
Li X M, Hedlund P B, Fuxe K. (1995) Cholecystokinin octapeptide in vitro and ex vivo strongly modulates striatal dopamine D2 receptors in rat forebrain sections. Eur J Neurosci. 7(5): 962-971.
Lines C, Challenor J, Traub M. (1995) Cholecystokinin and anxiety in normal volunteers: an investigation of the anxiogenic properties of pentagastrin and reversal by the cholecystokinin receptor subtype B antagonist L-365,260. British Journal of Clinical Pharmacology. 39(3): 235-242.
Lodge D J, Lawrence A J. (2001) Comparative analysis of the central CCK system in Fawn Hooded and Wistar Kyoto rats: extended localisation of CCK-A receptors throughout the rat brain using a novel radioligand. Regul Pept. 99(2-3): 191-201.
Lu L, Zhang B, Liu Z, Zhang Z. (2002) Reactivation of cocaine conditioned place preference induced by stress is reversed by cholecystokinin-B receptors antagonist in rats. Brain Research. 954(1): 132-140.
Lu L, Huang M, Ma L, Li J. (2001) Different role of cholecystokinin CCK-A and CCK-B receptors in relapse to morphine dependence in rats. Behavioral Brain Research. 120(1): 105-110.
Mackey W B, van der Kooy D. (1985) Neuroleptics block the positive reinforcing effects of amphetamine but not of morphine as measured by place conditioning. Pharmacol Biochem Behav. 22(1): 101-5.

Maidment N T, Siddall B J, Rudolph V R, Erdelyi E, Evans C J (1991) Dual determination of extracellular cholecystokinin and neurotensin fragments in rat forebrain: microdialysis combined with a sequential multiple antigen radioimmunoassay. Neuroscience. 45(1): 81-93.
Marshall F H, Barnes S, Hughes J, Woodruff G N, Hunter J C. (1991) Cholecystokinin modulates the release of dopamine from the anterior and posterior nucleus accumbens by two different mechanisms. J. Neurochem. 56(3): 917-922.
McCleane G J. (2002) A phase 1 study of the cholecystokinin (CCK) B antagonist L-365, 260 in human subjects taking morphine for intractable non-cancer pain. Neuroscience Letters. 332(3): 210-212.
McCleane G J. (2003) A randomised, double blind, placebo controlled crossover study of the cholecystokinin 2 antagonist L-365, 260 as an adjunct to strong opioids in chronic human neuropathic pain. Neuroscience Letters. 338(2): 151-154.
McLaughlin J, See RE. (2003) Selective inactivation of the dorsomedial prefrontal cortex and the basolateral amygdala attenuates conditioned-cued reinstatement of extinguished cocaine-seeking behavior in rats. Psychopharmacology. 168(1-2): 57-65.
Mercer L D, Beart P M (2004) Immunolocalization of CCK1R in rat brain using a new anti-peptide antibody. Neurosci Lett. 359(1-2): 109-13.
Meyer D K, Beinfeld M C, Oertel W H, Brownstein M J (1982) Origin of the cholecystokinin containing fibers in the caudate-putamen. Science. 215:187-188.
Morino P, Mascagni F, McDonald A, Hokfelt T (1994) Cholecystokinin corticostriatal pathway in the rat: evidence for bilateral origin from medial prefrontal cortical areas. Neuroscience. 59: 939-952.
Murphy R B, Schuster D I. (1982) Modulation of [3H]-dopamine binding by cholecystokinin octapeptide (CCK-8). Peptides. 3(3): 539-543.
Murphy M G, Sytnik B, Kovacs T O, Mertz H, Ewanik D, Shingo S, Lin J H, Gertz B J, Walsh J H. (1993) The gastrin-receptor antagonist L-365,260 inhibits stimulated acid secretion in humans. Clin Pharmacol Ther. 54(5): 533-539.
Nemeroff C B, Osbahr A J, Bissette G, Jahnke G, Lipton M A, Prange A J (1978) Cholecystokinin inhibits tail pinch-induced eating in rats. Science. 200(4343): 793-794.
Panerai A E, Rovati L C, Cocco E, Sacerdote P, Mantegazza P. (1987) Dissociation of tolerance and dependence to morphine: a possible role for cholecystokinin. Brain Research. 410(1): 52-60.
Phillips P E, Hancock P J, Stamford J A. (2002) Time window of autoreceptor-mediated inhibition of limbic and striatal dopamine release. Synapse. 44(1): 15-22.
Schanzer M C, Jacobson E D, Dafny N. (1978) Endocrine control of appetite: gastrointestinal hormonal effects on CNS appetitive structures. Neuroendocrinology. 25(6): 329-342.
Seroogy K B, Dangaran K, Lim S, Haycock J W, Fallon J H. (1989) Ventral mesencephalic neurons containing both cholecystokinin- and tyrosine hydroxylase-like immunoreactivities project to forebrain regions. J Comp Neurol. 279(3): 397-414.
Shippenberg T S, Herz A. (1988) Motivational effects of opioids: influence of D-1 versus D-2 receptor antagonists. Eur J Pharmacol. 151(2): 233-242.
Singh L, Lewis A S, Field M J, Hughes J, Woodruff G N (1991) Evidence for an involvement of the brain cholecystokinin B receptor in anxiety. Proceedings of the National Academy of Sciences. 88(4): 1130-1133.
Sramek J J, Kramer M S, Reines S A, Cutler N R. (1994) Pilot study of a CCK_{B} antagonist in patients with panic disorder: preliminary findings. Anxiety. 1(3): 141-143.
Tang J, Chou J, ladarola M, Yang H Y, Costa E. (1984) Proglumide prevents and curtails acute tolerance to morphine in rats. Neuropharmacology. 23(6): 715-718.
Tanganelli S, Fuxe K, Antonelli T, O'Connor W T, Ferraro L. (2001) Cholecystokinin/dopamine/GABA interactions in the nucleus accumbens: biochemical and functional correlates. Peptides. 22(8):1229-1234.
Tzschentke T M (1998) Measuring reward with the conditioned place preference paradigm: a comprehensive review of drug effects, recent progress and new issues. Prog Neurobiol. 56(6): 613-672.
Vaccarino F J, Vaccarino A L (1989) Antagonism of cholecystokinin function in the rostral and caudal nucleus accumbens: differential effects on brain stimulation reward. Neurosci Lett. 97(1-2):151-156.
van Megen H J, Westenberg H G, den Boer J A. (1996) Effect of the cholecystokinin-B receptor antagonist L-365,260 on lactate-induced panic attacks in panic disorder patients. Biol Psychiatry. 40(8): 804-806.
Voigt M, Wang R Y, Westfall T C. (1986) Cholecystokinin octapeptides alter the release of endogenous dopamine from the rat nucleus accumbens in vitro. J Pharmacol Exp Ther. 237(1): 147-153.
Wang R Y, Hu X T. (1986) Does cholecystokinin potentiate dopamine action in the nucleus accumbens? Brain Res. 380(2): 363-367.
Wang R Y, White F J, Voigt M M. (1985) Interactions of cholecystokinin and dopamine in the nucleus accumbens. Ann N Y Acad. Sci. 448: 352-360.
Watkins L R, Kinscheck I B, Mayer D J. (1984) Potentiation of opiate analgesia and apparent reversal of morphine tolerance by proglumide. Science. 224(4647): 395-396.
Weiss F, Maldonado-Vlaar C S, Parsons L H, Kerr T M, Smith D L, Ben-Shahar O. (2000) Control of cocaine-seeking behavior by drug-associated stimuli in rats: effects on recovery of extinguished operant-responding and extracellular dopamine levels in amygdala and nucleus accumbens. Proc Natl Acad Sci 97(8): 4321-4326.
White F J, Wang R Y. (1984) Interactions of cholecystokinin octapeptide and dopamine on nucleus accumbens neurons. Brain Res. 300(1): 161-166.
Willis G L, Hansky J, Smith G C. (1986) Central and peripheral proglumide administration and cholecystokinin-induced satiety. Regulatory Peptides. 15(1): 87-98.
Yim C C, Mogenson G J. (1991) Electrophysiological evidence of modulatory interaction between dopamine and cholecystokinin in the nucleus accumbens. Brain Res. 541(1): 12-20.
You Z-B, Herrera-Marschitz M, Brodin E, Meana J J, Morino P, Hokfelt T, Silveira R, Goiny M, Ungerstedt U (1994) On the origin of striatal cholecystokinin release: studies with in vivo microdialysis. J Neurochem. 62: 76-85.
You Z B, Tzschentke T M, Brodin E, Wise R A. (1998) Electrical stimulation of the prefrontal cortex increases cholecystokinin, glutamate, and dopamine release in the nucleus accumbens: an in vivo microdialysis study in freely moving rats. J Neurosci. 18(16): 6492-6500.
Yun I A, Wakabayashi K T, Fields H L, Nicola S M. (2004) The ventral tegmental area is required for the behavioral and nucleus accumbens neuronal firing responses to incentive cues. J Neurosci. 24(12): 2923-33.
Zajac J M, Gully D, Maffrand J P. (1996) [3H]-SR 27897B: a selective probe for autoradiographic labelling of CCK-A receptors in the brain. J Recept Signal Transduct Res. 16(1-2): 93-113.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of mitigating a symptom of maladaptive substance use, the method comprising systemically administering an effective amount of an inhibitor of a cholecystokinin-B (CCK_{B}) receptor to a subject, whereby the symptom of maladaptive substance use is mitigated.

2. The method of claim 1, wherein the substance comprises a drug selected from the group consisting of an opioid, a psychostimulant, a cannabinoid, an empathogen, a dissociative drug, and ethanol.

3. The method of claim 1, where the substance comprises food.

4. The method of claim 1, wherein the CCK_{B} receptor inhibitor comprises a CCK_{B} receptor antagonist that inhibits a function of the CCK_{B} receptor.

5. The method of claim 4, wherein the CCK_{B} receptor antagonist is selective for the CCK_{B} receptor.

6. The method of claim 5, wherein the CCK_{B} receptor antagonist comprises (3R-(+)-2,3-dihydro-1-methyl-2-oxo-5-phenyl-1H-1,4-benzodiazepin-3-yl)-N′-(3-methylphenyl)urea (L-365,260).

7. The method of claim 1, wherein the symptom of maladaptive substance use is selected from the group comprising: drug reward, incentive salience for a drug, drug craving, drug seeking, drug consumption, drug reinstatement, stress-induced reinstatement, and drug relapse.

8. The method of claim 1, comprising coadministering an inhibitor of an opioid receptor with the CCK_{B} receptor inhibitor.

9. The method of claim 8, wherein the amount of the opioid receptor inhibitor administered is sufficient to reduce the risk of drug overdose in the subject.

10. The method of claim 8, wherein the opioid receptor inhibitor comprises an opioid receptor antagonist that inhibits a function of the opioid receptor.

11. The method of claim 8, wherein the opioid receptor comprises the mu opioid receptor.

12. The method of claim 8, wherein the opioid receptor inhibitor comprises an opioid receptor antagonist that is selective for the mu opioid receptor.

13. The method of claim 12, wherein the selective mu opioid receptor antagonist comprises an antagonist selected from the group consisting of naltrexone, naloxone, CTAP, and CTOP.

14. The method of claim 1, wherein the CCKB receptor inhibitor is administered via implantation of a depot formulation comprising the inhibitor.

15. A pharmaceutical composition comprising:
(a) an inhibitor of a cholecystokinin-B (CCKB) receptor; and
(b) an inhibitor of an opioid receptor.

16-24. (canceled)

25. A method of prescreening for an agent that can modulate a symptom of maladaptive substance use in a subject, the method comprising:
(a) contacting a test agent with a cholecystokinin-B (CCKB) receptor; and
(b) determining whether the test agent specifically binds to the CCKB receptor; and
(c) if the test agent specifically binds to the CCKB receptor, selecting the test agent as a potential modulator of a symptom of maladaptive substance use in a subject.

26. (canceled)

27. A method of prescreening for an agent that can modulate a symptom of maladaptive substance use in a subject, the method comprising:
(a) contacting a test agent with a polynucleotide encoding a cholecystokinin-B (CCKB) receptor; and
(b) determining whether the test agent specifically binds to the polynucleotide encoding the CCKB receptor; and
(c) if the test agent specifically binds to the polynucleotide encoding the CCKB receptor, selecting the test agent as a potential modulator of a symptom of maladaptive substance use in a subject.

28. (canceled)

29. (canceled)

30. A method of screening for an agent that can modulate a symptom of maladaptive substance use in a subject, the method comprising:
(a) contacting a test agent with a cholecystokinin-B (CCKB) receptor;
(b) determining whether the test agent acts as an agonist or an antagonist of the CCKB receptor;
(c) if the test agent acts as an agonist or antagonist of the CCKB receptor, selecting the test agent as a potential modulator of maladaptive substance use in a subject.

31-40. (canceled)

41. A method of screening for an agent that that can modulate maladaptive substance use in a subject, the method comprising:
(a) selecting a modulator of a cholecystokinin-B (CCKB) receptor as a test agent; and
(b) measuring the ability of the selected test agent to modulate drug reward in an animal model.

42. (canceled)

43. (canceled)

44. A method of assessing a subject's risk for maladaptive substance use, the method comprising determining the level of cholecystokinin-B (CCKB) receptor polypeptides, polynucleotides, or function in a biological sample from the subject, wherein risk for maladaptive substance use is directly correlated with said level.

45. (canceled)

46. A kit comprising:
(a) an inhibitor of a cholecystokinin-B (CCKB) receptor in a pharmaceutically acceptable carrier;
(b) instructions for carrying out the method of claim 1.

47. A diagnostic kit comprising:
(a) a component that specifically binds to a cholecystokinin-B (CCKB) receptor polypeptide or polynucleotide; and
(b) instructions for carrying out the method of claim 44.
